(19) United States
(12) Patent Application Publication
(10) Pub. No.: US 2014/0074062 A1
(43) Pub. Date: Mar. 13, 2014

(54) PISTON PUMP DEVICES

(71) Applicants: Sean Caffey, Hawthorne, CA (US); Alice Lai, Pasadena, CA (US); Po-Ying Li, Monrovia, CA (US)

(72) Inventors: Sean Caffey, Hawthorne, CA (US); Alice Lai, Pasadena, CA (US); Po-Ying Li, Monrovia, CA (US)

(21) Appl. No.: 13/960,470

(22) Filed: Aug. 6, 2013

Related U.S. Application Data
(60) Provisional application No. 61/680,128, filed on Aug. 6, 2012.

(51) Int. Cl. A61M 5/42; A61M 5/142; A61M 5/155; A61M 5/19

(52) U.S. Cl. CPC A61M 5/422; A61M 5/19; A61M 5/14216; A61M 5/155
USPC 604/506; 604/82; 604/152; 604/67; 604/143; 29/888.02; 604/112

(57) ABSTRACT

Drug pump devices with syringe or pen-injection configurations may utilize pre-filled drug vials or cartridges; the prefilled vials may be equipped with mechanisms for stirring their contents and/or changing a chemical environment therein to improve therapies. To facilitate combination therapies, multiple drug pump devices may be assembled into a larger system. Lancet insertion devices for use in conjunction with the drug pump devices may feature improved safety characteristics and/or mechanisms for minimizing pain and discomfort.

### CROSS-REFERENCE TO RELATED APPLICATIONS

The present application claims priority to and the benefit of, and incorporates herein by reference in its entirety, U.S. Provisional Patent Application No. 61/680,128, filed on Aug. 6, 2012.

### TECHNICAL FIELD

The present invention relates generally to systems and methods for drug delivery. More specifically, various embodiments are directed to pre-filled syringe or piston pumps and methods for their manufacture and use.

### BACKGROUND

The treatment of many diseases requires regular subcutaneous or intramuscular drug injections. For example, diabetes patients may need insulin injections following every meal and, in addition, a continuously administered low “basal” rate of insulin. The major technologies currently in use for frequent or continuous drug delivery are syringes, pre-filled pen injectors, and patient-filled portable drug pump devices. Each of these technologies has disadvantages. For example, syringes, unless filled by a well-trained and skilled person (e.g., a health-care professional), can easily trap bubbles during the filling process, posing a risk to patient safety. Further, certain therapies require injection volumes greater than 1 ml; protein solutions, for example, often cannot be formulated at high concentration because the proteins will precipitate, so large-volume injections are employed instead. Large injection volumes, however, generally cannot be administered by syringe due to the risk of pain and swelling.

Pre-filled pen injectors are advantageous in that they facilitate accurate manual dosing using a pre-filled, bubble-free glass cartridge, which simplifies the priming process. Further, they allow injections to be administered quickly, which can reduce congestion is busy hospital settings and, in emergencies requiring immediate injection (such as an allergic reaction), even save lives. For routine self-administration of a drug (such as insulin) by the patient, however, they can pose several problems. Since the injection is performed manually, deficient patient compliance (e.g., improper injection timing and/or failure to follow the dosing prescription) is a major concern. In addition, improper or sub-optimal needle insertion or retraction can cause unnecessary pain and discomfort or, worse, be dangerous to the patient. Many standard pen injectors, for example, are designed to be activated by pushing a button at the top of the device, e.g., with the index finger, in order to deliver the dose after the needle has pierced the skin. This requires the patient to hold the device between his middle finger and thumb, or alternatively to grasp the device with his hand while depressing the button with his thumb, neither of which constitutes an optimal grip. As a result, the patient may not be able to insert the needle at a velocity and angle of entry that minimize pain, or may even miss the targeted injection site and accidentally hit, e.g., a nerve or vein. For intramuscular injections, which may be used to deliver drugs different from or concentrated higher than those injected subcutaneously, retracting the needle following drug delivery can likewise be dangerous, as any residual drug in the needle tip may drip into the subcutaneous tissue.

Further, many pen injectors include a needle-insertion mechanism that quickly advances the needle upon actuation of a trigger mechanism, such as a button. If the injector is not handled with care, the insertion mechanism may inadvertently be triggered before the injector is properly placed on the skin, risking injury or damage to people and objects in the environment and wasting the injector, which is, typically, designed for one-time use. Further, the drug in pre-filled cartridges may deteriorate over time due to sub-optimal storage conditions (e.g., an unsuitable pH), or—if measures are taken to improve shelf life—it may be unsuited for certain desirable routes of administration. For example, a drug formulated for storage in a very acidic or very alkaline environment may be unsuitable for subcutaneous injection. In addition, certain formulations may suffer from undesirable chemical precipitation, including, e.g., the separation of two or more components of a mixture of therapeutic agents.

Portable drug pump devices can provide fully controlled drug delivery over extended periods of time (e.g., days); therefore, patient compliance is much improved. Decreased numbers of needle insertions (once every three days, for example, rather than five times per day as is typical for syringe or pen injections) and programmable dosing schedules may greatly enhance the patient's quality of life. In addition, many portable pump devices are provided in the form of patch pumps with low pump profiles, which can be attached to the patient's skin without interfering with daily activities such as showering, sleeping, and exercising. However, filling standard portable pump devices is generally a time-consuming and intricate process, and since they are typically filled by patients, risks arise during the priming procedure. Improperly primed reservoirs may contain large air bubbles and cause the pump to inject too much air into the subcutaneous tissue, which is a serious safety matter. Further, with pump devices worn by the patient, the site of needle insertion is often hidden from view by the device housing, potentially allowing blood inside or around the cannula, or another problem associated with the needle insertion or subsequent shifting of the catheter, to go unnoticed. Additionally, since the drug reservoir of a portable pump device generally stores multiple dosage volumes, an inherent risk exists for overdosing an individual drug delivery.

In addition to the various potential shortcomings of pen injectors and patient-filled portable pump devices, currently available devices in both classes are generally limited to drug therapies involving only one drug formulation (which may include one or more therapeutic agents); more complex therapies, however, may involve the sequential application of multiple agents.

Accordingly, a need exists for drug delivery devices that are safer, easier, and as painless as possible to use for the patient, and that preferably facilitate more complex drug therapies.

### SUMMARY

The present invention provides, in various embodiments, drug pump devices that incorporate pre-filled drug vials or cartridges in a syringe or pen-injection configuration, which generally includes a linear arrangement of a drug reservoir, a piston movable within the reservoir, and a pump (e.g., an electrolysis pump) for driving the piston towards an outlet of the reservoir so as to expel drug. Such drug pump devices are hereinafter called “syringe pump devices” or “piston pump devices.” (A “syringe pump” may refer, specifically, to a device in which the piston is actuated by a mechanical member (serving as the “pump”), whereas the term “piston pump” is usually used in reference to devices driven by a different force, such as pressure in a pump chamber. However, for purposes of the following description, the terms are generally used interchangeably.) For use in conjunction with drug pump devices, various embodiments further provide needle or lancet insertion devices with improved safety characteristics and/or features for minimizing pain and discomfort. Certain embodiments automate needle insertion and/or pump operation to minimize the mechanical components to be operated by the patient and thereby increase the ease of use of the devices.

In some embodiments, two or more syringe pump devices are combined in a larger assembly, facilitating complex drug therapies that involve, e.g., two or more therapeutic agents that need to be stored separately, a therapeutic agent that requires activation by an activating agent just prior to insertion, or a therapeutic agent and a pharmacokinetic agent that affects the local infusion environment. Fluidic paths from the outlets of the drug reservoirs of the respective pump devices may merge prior to reaching the injection site, e.g., in a mixing chamber. For drug pump assemblies that include three or more separate pump devices, the contents of the respective reservoirs may be mixed sequentially utilizing multiple staged mixing chambers. Check valves or active valves in the fluidic paths may serve to prevent backflow from the injection site and/or to control the flow rate from various reservoirs towards the injection site. In alternative embodiments, multi-pump assemblies are used to achieve very accurate drug dosing for a single formulation and, in particular, prevent overdosing. Each drug pump device of the assembly may store drug in the amount needed for a single bolus delivery. Multiple doses may then be administered by separately and sequentially injecting the contents of the various reservoirs.

In some embodiments, the pre-filled drug pump device and the lancet insertion device are integrated with one or more skin-adhesive patches, forming a wearable drug delivery device (or “patch pump device”) that facilitates controlled drug delivery in multiple doses and/or over an extended period of time with a single lancet insertion while eliminating the need for the patient (or a healthcare provider or other person assisting the patient) to fill the device herself. The lancet insertion device and/or surrounding patch may have a window (e.g., formed by a cut-out in the housing and/or transparent material) that is positioned so as to allow visual inspection of the injection site, alerting the patient when the device should be repositioned or replaced.

In some embodiments, the pre-filled drug pump device and the lancet insertion device are integrated into a handheld pen injector, which is typically intended for one-time use and disposable. The pen injector may be equipped with a sensor for detecting skin contact that is placed, e.g., on or near the tip of the lancet or on the underside of the insertion device in the vicinity of the insertion site. The sensor may serve as a safety interlock that prevents inadvertent “firing” of the lancet until skin contact is established. The same or another sensor, when located on or near the lancet tip, may also be used to determine whether the lancet has penetrated the skin and entered subcutaneous tissue and, if so, automatically activate the pump to initiate drug delivery. This obviates the need for the patient to press a button or actuate a similar mechanical trigger, easing the convenience of use and allowing the patient, for instance, to hold the pen injector between thumb and index finger, which, in turn, enables a more confident stab. As a result, the patient may be able to orient the lancet for pain minimization, e.g., at a 45° angle with the beveled edge down. Although particularly advantageous in pen-injector embodiments, the sensor described above may also be used in conjunction with wearable (e.g., patch pump) devices. Further, certain pen injector devices are encased in a manner that normally obstructs viewing of the needle injection site, rendering a window feature, as described above for patch pump devices, desirable.

The pain experienced by the patient may also be reduced, in both patch-pump and pen-injector embodiments, by means of a distraction mechanism that provides a mechanical (or other) stimulus to the patient just (e.g., less than one second) prior to lancet insertion. For instance, certain devices may include a vibrator, an elastic band snapping the skin, a pincher, or a mechanism for releasing a small amount of cold water, to name just a few possibilities. The relative timing of the distracting stimulus and the subsequent lancet insertion may be controlled electronically, or be effected mechanically, e.g., via a needle-insertion trigger mechanism that is released by the distracting mechanism.

In electrolytically driven syringe pump devices, electrolysis gas is generated from an electrolyte contained within a pump chamber adjacent the piston, and gas pressure drives the piston forward. In certain embodiments, a spark inside the pump chamber, or some other mechanism, is used to reverse this process, i.e., to recombine the electrolysis gas to reduce the pressure in the pump chamber below that in the reservoir and thereby create suction at the reservoir outlet. This suction may be used during retraction of the needle or catheter from the patient to avoid dripping of residual fluid into the subcutaneous tissue. In some embodiments, suction is moreover used to draw blood or other fluid from the patient, e.g., for diagnostic purposes.

Syringe pump devices in accordance with various embodiments may be manufactured from pre-fabricated components. For instance, a piston fitted to the interior diameter of a glass vial may be inserted into the open end of the vial (the other end being plugged by a puncturable septum), and a pump assembly may thereafter be slid into or over, or otherwise mounted to, the open end of the vial to thereby close it. The pump assembly may, e.g., include electrolysis electrodes and associated driver and control circuitry. The drug reservoir and pump chamber created in the vial to both sides of the piston may be filled with a needle inserted into the pump device either through the drug reservoir septum or through a puncturable plug integrated with the pump assembly that defines the back end of the pump chamber. In some embodiments, a needle structure with two axial bores of different lengths is utilized to simultaneously fill the drug reservoir with the liquid medication and the pump chamber with electrolyte, which saves time in the manufacturing process. In certain embodiments, a fill port penetrating the pump plug and the piston is permanently integrated with the device, providing fluidic paths for re-filling both the drug reservoir and the pump chamber as needed. To aid inexpensive mass manufacturing of drug pump devices in accordance herewith, certain devices components may be provided in standard sizes and/or configurations. For example, various devices may utilize standard glass or polymer vials holding up to 3 ml of liquid drug (leaving sufficient space for the piston and pump chamber). In order to decrease the total volume of the drug reservoir (i.e., the starting volume prior to drug delivery) without increasing the volume of the pump chamber (which would lead to decreased pump pressures or increased power requirements to achieve the same pump pressure), a spacer may be inserted between the piston and pump chamber. On the other hand, to achieve total drug volumes exceeding 3 ml, e.g., as often used for intramuscular injections, multiple 3-ml drug pump devices may be combined into one drug assembly.

Drug pump devices in accordance with various embodiments are designed for long storage periods (e.g., weeks, months, or even years). To increase the shelf life of the devices, the chemical environment in the drug reservoir may be optimized for drug stability, for example, by adjusting the pH. The optimal storage conditions do not, however, always coincide with the optimal delivery conditions. Therefore, certain drug pumps in accordance herewith are equipped with an electrode pair inside the drug reservoir. Electricity applied to the electrodes may be used to activate the drug, change the pH of the solution, or otherwise alter the chemical or physicochemical environment in the reservoir. Furthermore, to create uniform distribution of the components of a mixture and/or reverse any precipitation, the reservoir may be equipped with a magnetic (or other) stirring mechanism. For example, the reservoir may be surrounded (at least partially) by an electromagnetic sleeve or coil and contain a magnetic stirrer that can be magnetically activated prior to drug injection to mix the contents of the reservoir.

Accordingly, in a first aspect, the invention pertains to a drug pump assembly including two piston pump devices; each piston pump device includes a vial having a drug reservoir therein, a piston movable within the vial for forcing drug out of an outlet of the reservoir, and a pump (e.g., an electrochemical pump, an osmotic pump, an electro-osmotic pump, a piezoelectric pump, a thermo-pneumatic pump, an electrostatic pump, a pneumatic pump, an electro-hydrodynamic pump, a magneto-hydrodynamic pump, an acoustic-streaming pump, an ultrasonic pump, and/or an electrically driven mechanical pump) for actuating the piston. In various embodiment, the drug pump assembly includes a first mixing chamber downstream of the reservoirs, first set of fluid conduits (e.g., tubing, channels, etc.) connecting the outlets of the reservoirs with the first mixing chamber, and a second fluid conduit connecting the first mixing chamber with a drug delivery vehicle downstream thereof.

In some embodiments, the drug pump assembly includes a third piston pump device having a vial that has a drug reservoir therein, a piston movable within the vial for forcing drug out of an outlet of the reservoir, and a pump mechanism for actuating the piston. The assembly may include a third fluid conduit connecting the outlet of the third reservoir of the third piston pump device with the mixing chamber. In one implementation, the assembly includes a second mixing chamber downstream of the first mixing chamber and upstream of the drug delivery vehicle; the third fluid conduit may then connect the outlet of the third piston pump to the second mixing chamber, and the second fluid conduit may connect the outlet of the first mixing chamber to the second mixing chamber and the second mixing chamber to the drug delivery vehicle. The first mixing chamber and/or the second mixing chamber may include a stirring mechanism (e.g., a pump, a fan, a turbine, or magnets).

Additionally, the assembly may include one or more valves between one of the reservoir outlets and/or the first mixing chamber. The valves may be or include, for example, a check valve preventing backflow or an active valve regulating fluid flow. The assembly may further include one or more sensors disposed within one or more drug reservoirs and/or the fluid conduits for monitoring one or more parameters therein, and a controller for controlling the valve(s) based on the monitored parameter(s).

In another aspect, the invention relates to a method for treating a target using an assembly having two piston pump devices (each piston pump including a vial having a drug reservoir therein, a piston movable within the vial for forcing drug out of an outlet of the reservoir, and a pump for actuating the piston). In various embodiments, the method includes actively mixing liquids released from the drug reservoirs of the two piston pump devices in a mixing chamber and delivering the mixed liquid to the target via fluid conduits.

The method may further include monitoring one or more parameters of the liquids in the piston pump devices and regulating flows of the liquids based on the monitored parameter(s). Additionally, the method may include reducing the pressure in one or more piston pump devices below that of the respective reservoir so as to create suction and thereby prevent the mixed liquid from infiltrating the target or induce the mixed liquid to flow in a direction from the target site to the piston pump devices. The pump may be an electrolysis pump generating electrolysis gas within the pump chamber in mechanical contact with the piston, and the pressure reduction may be achieved using a mechanism for recombining the electrolysis gas.

Another aspect of the invention relates to a method for treating a target using a drug pump assembly having two drug reservoirs fluidically connectable to the injection site in the target. In some embodiments, the method includes providing a first therapeutic fluid from the first reservoirs to the target and subsequently providing a second therapeutic fluid, different from the first therapeutic fluid, from the second reservoir to the target. The first therapeutic fluid may pharmacokinetically affect a local environment of the target, and the second therapeutic fluid includes an active ingredient for treating the target.

In yet another aspect, a method for treating a target using a drug pump assembly that has two drug reservoirs and a mixing chamber includes delivering a first therapeutic fluid from the first reservoir to the mixing chamber; providing the second therapeutic fluid, different from the first therapeutic fluid, from the second reservoir to the mixing chamber; mixing the first and second therapeutic fluids in the mixing chamber; and delivering the mixed first and second therapeutic fluids to the target. In some embodiments, the first therapeutic fluid includes an active ingredient for treating the target and the second therapeutic fluid activates the active ingredient of the first fluid.

Still another aspect of the invention relates to a clinical trial method using a drug pump assembly having two drug reservoirs. In some embodiments, the method includes delivering the therapeutic fluid from the first reservoir to a target within a patient and measuring a response of the target thereto; delivering a physiological saline solution from the second reservoir to the target and measuring a response of the target thereto; and comparing the responses of the target to the therapeutic fluid and the physiological saline solution, respectively, to thereby determine an effect of the therapeutic fluid.

In another aspect, the invention relates to a drug pump assembly. In various embodiments, the assembly includes multiple drug pump devices, each having a drug reservoir holding a specified dosage of liquid drug, and control circuitry for operating the drug pump devices so as to deliver liquid drug sequentially from the devices to a delivery vehicle according to a delivery protocol; delivery from each of the devices includes delivery of substantially (e.g., at least 90%, preferably at least 95%, or even at least 99% of) the entire specified dosage held therein. In one embodiment, the drug pump devices are piston pump devices, each having a vial defining the drug reservoir therein, a piston movable within the vial for forcing drug out of an outlet of the reservoir, and a pump for actuating the piston.

In still another aspect, the invention relates to a method for delivering drug from multiple drug pump devices, each having a drug reservoir holding a specified dosage of liquid drug. In some embodiments, the method includes delivering liquid drug sequentially from the devices to a delivery vehicle according to a delivery protocol; delivery from each of the devices includes delivery of substantially the entire specified dosage held therein.

In still another aspect, the invention pertains to a drug pump device. In various embodiments, the device includes a drug reservoir containing liquid drug therein and having an outlet fluidically connected to a drug delivery vehicle; a displaceable member in mechanical contact with the reservoir for forcing drug out of the outlet of the reservoir; a pump for actuating the displaceable member; and a stirring mechanism for stirring the liquid drug prior to delivery via the drug delivery vehicle. In one implementation, the stirring mechanism is a magnetic stirring mechanism. For example, the stirring mechanism may include an electromagnet for generating an alternating magnetic field within the reservoir. The liquid drug may include nonmagnetic particles responsive to the alternating magnetic field. Alternatively or additionally, the stirring mechanism may further include, contained within the reservoir, a magnetic stirrer responsive to the alternating magnetic field. In some embodiments, the drug reservoir is formed within a vial, and the electromagnet at least partially surrounds the vial. In one embodiment, the electromagnet includes an electromagnetic sleeve or coil.

Further, the device may include electronic circuitry, responsive to a drug delivery initiation signal, for activating the stirring mechanism so as to stir the liquid drug and thereafter operating the pump to cause drug delivery. The drug delivery initation signal may be provided by a programmed drug delivery protocol and/or a manual trigger action.

In another aspect, the invention relates to a drug pump device. In various embodiments, the drug pump device includes a drug reservoir having a liquid that contains a therapeutic agent and an outlet fluidically connected to a drug delivery vehicle; a displaceable member in contact with the reservoir for forcing the liquid out of the reservoir outlet; a pump for actuating the displaceable member; and a pair of electrodes disposed within the reservoir for changing a chemical or physical environment therein. A voltage may be applied between the electrodes to cause activation of the therapeutic agent, a change in pH of the liquid, and/or a chemical reaction therein. In one implementation, the pump is an electrolysis pump fluidically isolated from the reservoir.

Another aspect of the invention relates to a method of manufacturing a drug pump device. In some embodiments, the method includes inserting a piston into the open end of a vial, the other end of the vial being plugged by a septum; placing an electrolysis pump assembly at the open end of the vial, thereby closing it; piercing the septum to establish the first fluidic path between the exterior of the device and a drug reservoir formed between the septum and the piston, and delivering liquid drug via the first fluidic path to the reservoir; and piercing the piston to establish the second fluidic path between the exterior of the device and a pump chamber formed between the piston and the pump assembly, the second path going through the reservoir, and delivering liquid electrolyte via the second fluidic path to the pump chamber.

The septum and piston may be pierced via a needle structure having two bores providing the first and second fluidic paths; the second outlet end of the second bore may be placed closer to a tip of the needle structure than the first outlet end of the first bore. The first outlet end may be placed inside the drug reservoir and the second outlet end may be simultaneously placed inside the pump chamber; the liquid drug and electrolyte may then be simultaneously delivered to the drug reservoir and pump chamber, respectively. In one embodiment, the method further includes withdrawing the needle from the device.

In yet another aspect, a method of manufacturing a drug pump device includes, in various embodiments, inserting a piston into the open end of a vial, thereby forming a drug chamber between the closed end of the vial and the piston; placing an electrolysis pump assembly at the open end of the vial, the pump assembly having a pierceable plug sealing the open end and forming a pump chamber between the plug and the piston; piercing the plug to establish the first fluidic path between the exterior of the device and the pump chamber, and delivering electrolyte via the first fluidic path to the pump chamber; and piercing the piston to establish the second fluidic path between the exterior of the device and the drug chamber, the second path going through the pump chamber, and delivering liquid drug via the second fluidic path to the drug chamber. In some embodiments, the plug and piston are pierced via a needle structure having two bores providing the first and second fluidic paths; the second outlet end of the second bore is placed closer to a tip of the needle structure than the first outlet end of the first bore. Additionally, the first outlet end is placed inside the pump chamber and the second outlet end is simultaneously placed inside the drug chamber; the liquid electrolyte and liquid drug are then simultaneously delivered to the pump chamber and drug chamber, respectively. In one implementation, the method further includes withdrawing the needle from the device.

Still another aspect of the invention relates to a drug pump device. In some embodiments, the device includes a vial closed by a first plug at the first end thereof; an electrolysis pump assembly placed at the second end of the vial and having a second plug sealing the second end; a piston movably disposed within the vial and dividing the vial into a drug chamber formed between the first plug and the piston and a pump chamber formed between the second plug and the piston; and a fill port penetrating the second plug and the piston and establishing a first fluidic path between the exterior of the device and the pump chamber and a second fluidic path between the exterior of the device and the drug chamber. The fill port may be affixed to the pump assembly and movable relative to the piston. Alternatively, the fill port may be affixed to the piston and movable relative to the pump assembly.

In another aspect, the invention relates to a drug delivery system. In some embodiments, the system includes a drug pump device; a lancet insertion device having a lancet and an insertion mechanism for driving the lancet from a retracted position to an extended position; a sensor for detecting contact with a patient's skin (e.g., a temperature or impedance sensor); and a user-operable lancet trigger mechanism for activating the insertion mechanism so as to cause insertion of the lancet into the patient. The trigger mechanism is responsive to the sensor and permits activation of the insertion mechanism only when the sensor detects contact with the skin.

The sensor may be located on the drug pump device and/or the lancet insertion device so as to be in contact with the patient's skin during drug delivery. Additionally, the sensor may be disposed on the tip of the lancet. Further, the sensor may be configured to detect insertion of the lancet into the patient. In various embodiments, the drug delivery system includes a pump trigger mechanism responsive to the sensor for automatically initiating pump operation upon the detection of lancet insertion.

In still another aspect, a drug delivery system includes a drug pump device; a lancet insertion device having a lancet and an insertion mechanism for driving the lancet from a retracted position to an extended position; a sensor for detecting insertion of the lancet into the patient; and a pump trigger mechanism responsive to the sensor for automatically initiating pump operation upon the detection of lancet insertion.

In another aspect, the invention pertains to a drug delivery system. In some embodiments, the system includes a drug pump device; a lancet insertion device having a lancet and an insertion mechanism for driving the lancet from a retracted position to an extended position; a distraction mechanism (e.g., a vibrator, a snapping elastic band, a pincher, a dull needle, and/or a mechanism for releasing cold water) for providing a mechanical, thermal, or other stimulus to a patient prior to insertion of the lancet; and a trigger mechanism for first triggering the distraction mechanism and, thereafter within a distraction period, triggering the insertion mechanism so as to cause insertion of the lancet while the patient is distracted.

The trigger mechanism may include circuitry for sending the first trigger signal to the distraction mechanism and, following a specified time delay, sending the second trigger signal to the insertion mechanism. Alternatively, the trigger mechanism may include a mechanical structure, activated by the distraction mechanism, for triggering the insertion mechanism.

In still another aspect, a drug delivery system includes a drug pump device having a drug reservoir; a lancet insertion device having a lancet and a cannula fluidically connected to the drug reservoir; and a housing at least partially enclosing the drug pump device and the lancet insertion device, the housing having a window therein allowing viewing of the cannula insertion site.

In yet another aspect, the invention relates to a method for delivering drug from an electrolytically driven drug pump device that has a drug reservoir fluidically connected to a drug delivery vehicle and a pump chamber in mechanical communication with the drug reservoir via an intervening displacement member. In various embodiments, the method includes inserting the drug delivery vehicle into subcutaneous tissue; electrolytically generating gas in the pump chamber, the gas exerting a pressure on the displacement member so as to drive liquid drug from the reservoir via the drug delivery vehicle into the tissue; causing recombination of the electrolytically generated gas to generate suction in the drug delivery vehicle; and while the suction persists, withdrawing the drug delivery vehicle from the tissue. The suction during withdrawal of the drug delivery vehicle may prevent drug from dripping out of the drug delivery vehicle.

### BRIEF DESCRIPTION OF THE DRAWINGS

The foregoing will be more readily understood from the following detailed description of the invention, in particular, when taken in conjunction with the drawings, in which:

FIG. 1A is a block diagram illustrating the various functional components of drug delivery systems in accordance with various embodiments;

FIG. 1B is an isometric view of a wearable piston pump device and infusion set in accordance with various embodiments;

FIGS. 1C and 1D are isometric and exploded views, respectively, of a drug delivery device with integrated lancet insertion mechanism in accordance with various embodiments;

FIG. 1E is a side view illustrating lancet insertion using the device of FIGS. 1C and 1D;

FIG. 1F is a side view of a pen-injection device in accordance with various embodiments;

FIG. 1G is a schematic side view of a piston pump device with a spark-ignition recombination mechanism in accordance with various embodiments;

FIGS. 2A and 2B are schematic views of drug pump assemblies including two pump devices and a mixing chamber in accordance with various embodiments;

FIG. 2C is a schematic view of a drug pump assembly including three pump devices and a mixing chamber in accordance with various embodiments;

FIG. 2D is a schematic view of a drug pump assembly including three pump devices and two staged mixing chambers in accordance with various embodiments;

FIGS. 2E and 2F are schematic views of different arrangements of the multiple pump devices of a drug pump assembly in accordance with various embodiments;

FIG. 2G is a schematic view of a drug pump assembly including multiple parallel drug pump devices in accordance with various embodiments;

FIG. 2H is a side view of a piston pump device with two pump chambers in a serial configuration in accordance with various embodiments;

FIGS. 3A and 3B are perspective views of a piston pump device including a spacer in accordance with various embodiments, during and after assembly, respectively;

FIGS. 4A and 4B are side views of piston pump devices illustrating filling thereof via a needle piercing the reservoir septum in accordance with various embodiments;

FIG. 4C is a schematic side view of a needle structure comprising two bores for simultaneously filling the drug reservoir and pump chamber of a piston pump device in accordance with various embodiments;

FIG. 4D is a schematic view of a filling system for a drug pump assembly including multiple pump devices in accordance with various embodiments;

FIG. 4E is a schematic view of a piston pump device with a refill port for filling the drug reservoir and pump chamber in accordance with various embodiments;

FIG. 5 illustrates a piston pump device with a magnetic stirring mechanism in accordance with various embodiments;

FIG. 6 illustrates a piston pump device containing electrodes in the pump reservoir for affecting a chemical environment therein in accordance with various embodiments;

FIGS. 7A and 7B illustrates the position of a skin sensor for different drug delivery devices in accordance with various embodiments;

FIG. 7C is a block diagram illustrating the functional components of lancet trigger and insertion mechanisms with a sensor-responsive safety interlock in accordance with various embodiments;

FIG. 8 illustrates a system for detecting needle insertion in accordance with various embodiments;

FIGS. 9A and 9B illustrate, respectively, a drug pump device without and a drug pump device with a window for viewing the injection site in accordance with various embodiments;

FIGS. 10A and 10B illustrate a drug pump device with a vibrator for distracting a patient during needle insertion in accordance with various embodiments; and

FIG. 10C illustrates a drug pump device with a rubber band for snapping the patient prior to needle insertion in accordance with various embodiments.

### DETAILED DESCRIPTION

### 1. Syringe Pump System Configurations

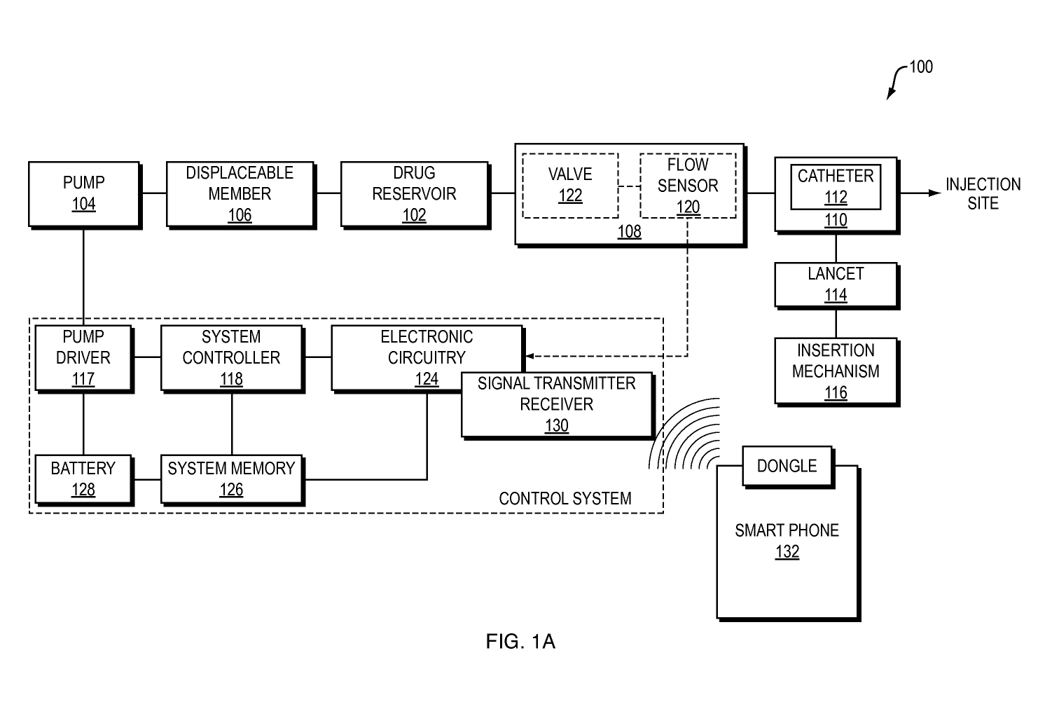

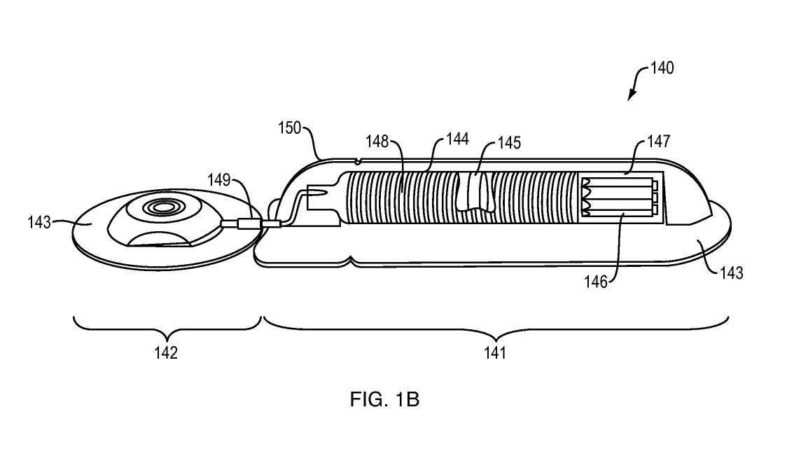

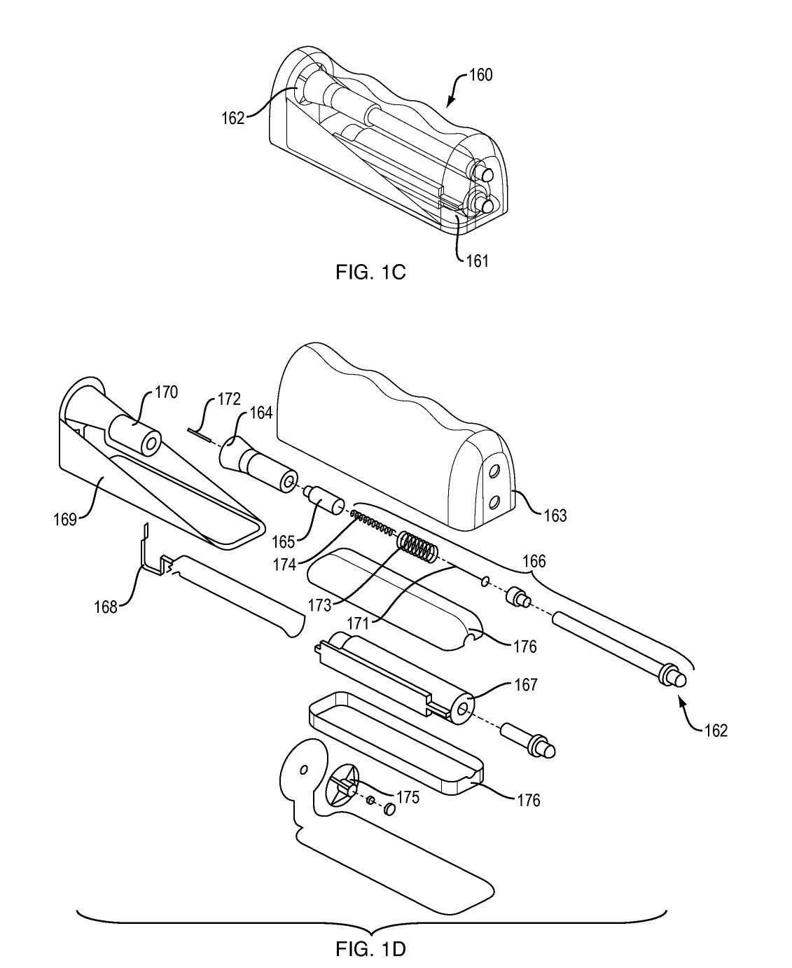

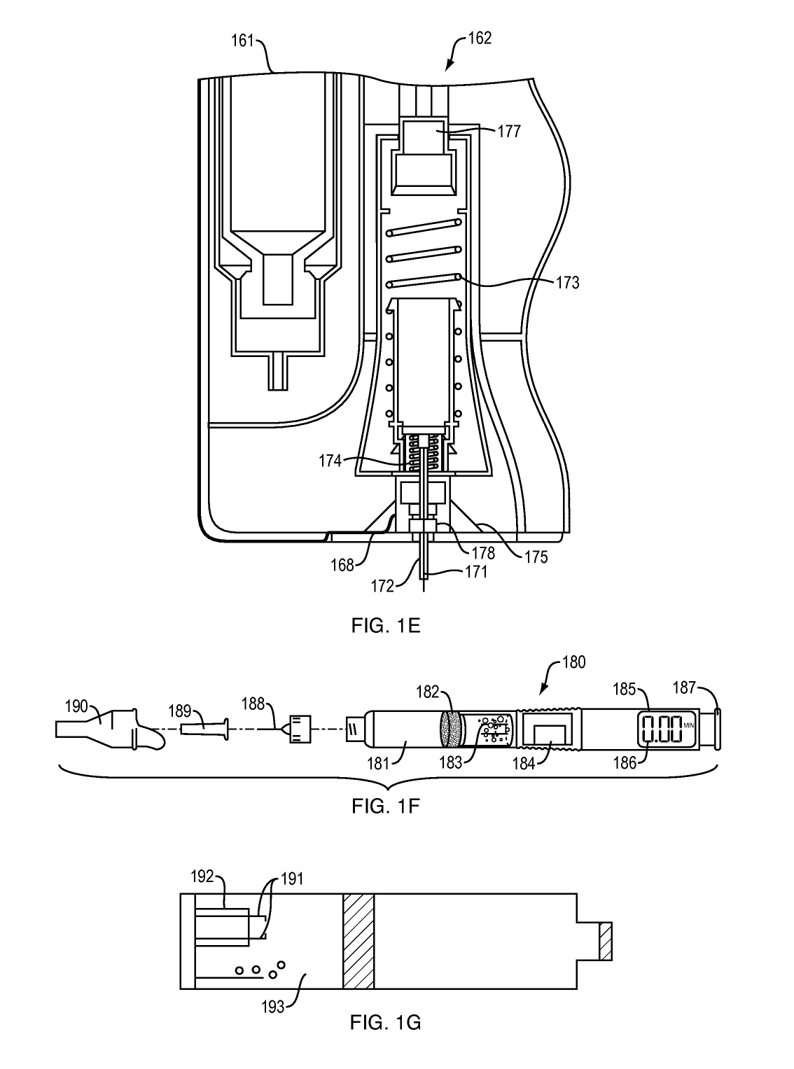

Various embodiments of the present invention provide pre-filled, stand-alone drug delivery assemblies with integrated lancet and/or catheter insertion mechanisms. FIG. 1A illustrates the major functional components of an exemplary such assembly in block-diagram form. The drug delivery assembly (or “system”) 100 includes a drug reservoir 102 that interfaces with a pump 104 via a displaceable member 106, such as a piston. In use, the drug reservoir 102 is filled with medication in liquid form, and pressure or a drive force generated by the pump 104 moves the displaceable member 106 so as to push the liquid drug out of the reservoir 102. Tubing 108 (e.g., a cannula made of medical-grade plastic) connected to an outlet of the drug reservoir 102 conducts the liquid to an infusion set 110. For wearable devices designed for multiple drug injections, the infusion set 110 may include a catheter 112 that is fluidically connected to the cannula 108 and delivers the drug to a subcutaneous tissue region. A lancet or needle 114 (hereinafter used interchangeably) and associated insertion mechanism 116 may be used to drive the catheter 112 through the skin. Following catheter insertion, the lancet or needle 114 may be retracted and removed from the device, leaving the soft catheter 112 in place. Alternatively, in some embodiments, the infusion set 110 includes another type of drug-delivery vehicle (i.e., device or device component in contact with and delivering the drug to the injection site), e.g., a sponge or other means facilitating drug absorption through the skin surface. In disposable devices for one-time use, the needle 114 itself may be fluidically connected to the cannula 112 and serve as the drug-delivery vehicle.

The pump 104 may drive the displaceable member 102 via manual or electrically driven mechanical actuation, by means of pressure (e.g., generated by gas release or chemical volume expansion) in a pump chamber adjacent the displaceable member 102, or using any other kind of pump mechanism. Suitable pumps include, for example, electrochemical, electrostatic, electrolytic, osmotic, electroosmotic, piezoelectric, thermopneumatic, electrostatic, pneumatic, electrohydrodynamic, magnetohydrodynamic, acoustic-streaming, ultrasonic, steam, lithium-outgassing, spring-loaded, gear motor, screw, vein, gear, and lobe pumps. In certain embodiments, electrolysis provides the mechanism that mechanically drives drug delivery. An electrolysis pump generally includes an electrolyte-containing chamber (the pump chamber) and, disposed in the chamber, one or more pairs of electrodes that are driven by a direct-current power source to break the electrolyte into gaseous products. Suitable electrolytes include water and aqueous solutions of salts, acids, or alkali, as well as non-aqueous ionic solutions. The electrolysis of water is summarized in the following chemical reactions:

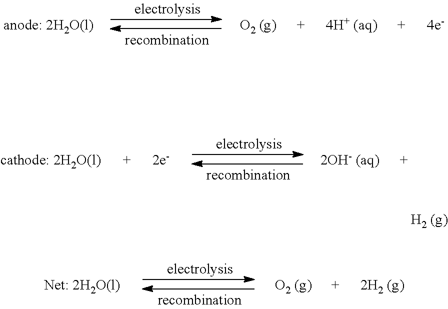

The net result of these reactions is the production of oxygen and hydrogen gas, which causes an overall volume expansion of the drug chamber contents. This gas evolution process proceeds even in a pressurized environment (reportedly at pressures of up to 200 MPa). As an alternative (or in addition) to water, ethanol may be used as an electrolyte, resulting in the evolution of carbon dioxide and hydrogen gas.

The pressure generated by the drug pump 104 may be regulated via a pump driver 117 by a system controller 118. For example, in an electrolytic pump, the controller 118 may set the drive current and thereby control the rate of electrolysis (which is generally proportional to the current), which, in turn, determines the pressure. Monitoring the pressure inside the pump chamber facilitates controlling the electrolysis current and duration so as to generate a desired volume of electrolysis gas, and thereby displace the same volume of liquid drug from the reservoir 102. In certain low-cost embodiments, the dose of drug to be delivered from the reservoir 102 is dialed into the device using a mechanical switch (e.g., a rotary switch), which then activates the pump 104, via the controller 118, to deliver the dose. In various alternative embodiments, the controller 118 executes a drug-delivery protocol programmed into the device or commands wirelessly transmitted to the device. In addition to controlling the drug pump 104, the controller 118 may be used to control other components of the drug delivery system 100; for example, it may operate the insertion mechanism 116 to trigger insertion of the lancet 114 and catheter 112. Alternatively, the lancet insertion mechanism 116 may be implemented using a separate controller or mechanical trigger mechanism.

The system controller 118 may be responsive to one or more sensors that measure an operational parameter of the drug delivery assembly 100, such as the pressure or flow rate in the drug reservoir 102 or cannula 108, the pressure inside the pump chamber, barometric pressure changes, temperature changes, or the position of the displaceable member 106. For example, the controller 118 may adjust the electrolysis based on the pressure inside the pump chamber, as described above; due to the inexpensiveness of pressure sensors, this option is particularly advantageous for pumps designed for quick drug delivery. As another example, if the patient walks on an airplane plane and the cabin begins to pressurize, this may alter the drug flow rate (e.g., by shrinking or expanding the electrolysis bubbles or changing pressure across the cannula). The controller 118 can recognize the change in environmental pressure, and may either alert the patient, or calculate the pressure and adapt the electrolysis current to achieve the desired flow rate. Two or more pressure sensors may be placed in the pump chamber to simultaneously monitor pressure therein, which provides additional feedback to the controller 118, improves accuracy of information, and serves as a backup in case of malfunction of one of the sensors. In general, the sensors used to measure various pump parameters may be flow, thermal, time of flight, pressure, or other sensors known in the art, and may be fabricated (at least in part) from parylene—a biocompatible, thin-film polymer. Multiple pressure sensors may be used to detect a difference in pressure and calculate the flow rate based on a known laminar relationship. In the illustrated embodiment, a flow sensor 120 (e.g., a MEMS sensor) is disposed in the cannula 108 to monitor drug flow to the infusion site, and detect potential obstructions in the flow path, variations in drug-pump pressure, etc. The cannula 108 may further include a valve 122, e.g., a check valve for preventing backflow of liquid into the drug reservoir 102, or an active valve that facilitates variably restricting the flow rate and, thus, provides an additional means of dosage control. Like the sensor 120, the valve 122 may be made of parylene. In other embodiments, silicon or glass are used in part for the flow sensor 120 and valve 122 construction. The sensor signals may be processed by electronic circuitry 124, which may, but need not, be integrated with the system controller 118.

The system controller 118 may be a microcontroller, i.e., an integrated circuit including a processor core, memory (e.g., in the form of flash memory, read-only memory (ROM), and/or random-access memory (RAM)), and input/output ports. The memory may store firmware that directs operation of the drug pump device. In addition, the device may include read-write system memory 126. In certain alternative embodiments, the system controller 112 is a general-purpose microprocessor that communicates with the system memory 126. The system memory 126 (or memory that is part of a microcontroller) may store a drug-delivery protocol in the form of instructions executable by the controller 118, which may be loaded into the memory at the time of manufacturing, or at a later time by data transfer from a hard drive, flash drive, or other storage device, e.g., via a USB, Ethernet, or firewire port. In alternative embodiments, the system controller 118 comprises analog circuitry designed to perform the intended function, e.g., to deliver the entire bolus upon manual activation by the patient.

The drug-delivery protocol may specify drug delivery times, durations, rates, and dosages, which generally depend on the particular application. For example, some applications require continuous infusion while others require intermittent drug delivery to the subcutaneous layer. An insulin-delivery system may be programmed to provide both a continuous, low basal rate of insulin as well as bolus injections at specified times during the day, typically following meals. Sensor feedback may be used in combination with a pre-programmed drug-delivery protocol to monitor drug delivery and compensate for external influences that may affect the infusion rate despite unchanged electrolysis (such as backpressure from the infusion site or cannula clogging). For example, signals from the flow sensor 120 may be integrated to determine when he proper dosage has been administered, at which time the system controller 118 terminates the operation of the pump 104 and, if appropriate, causes retraction of the delivery vehicle. The system controller 118 may also assess the flow through the cannula 108 as reported by the flow sensor 120, and take corrective action if the flow rate deviates sufficiently from a programmed or expected rate. If the system controller 118 determines that a higher flow rate of drug is needed, it may increase the current to the electrolysis electrodes to accelerate gas evolution in the electrolysis chamber; conversely, if the system controller 118 determines that a lower flow rate of drug is needed, it may decrease the current to the electrolysis electrodes.

The pump driver 110, system controller 118, and electronic circuitry 124 may be powered by a battery 128. Suitable batteries 128 include non-rechargeable lithium batteries approximating the size of batteries used in wristwatches, as well as rechargeable Li-ion, lithium polymer, thin-film (e.g., Li-PON), nickel-metal-hydride, and nickel cadmium batteries. Other devices for powering the drug pump system 100, such as a capacitor, solar cell or motion-generated energy systems, may be used either in place of the battery 128 or supplementing a smaller battery. This can be useful in cases where the patient needs to keep the drug-delivery system 100 on for several days or more.

In certain embodiments, the drug pump device 100 includes, as part of the electronic circuitry 124 or as a separate component, a signal receiver (for uni-directional telemetry) or a transmitter/receiver 130 (for bi-directional telemetry) that allows the device to be controlled and/or re-programmed remotely by a wireless handheld device, such as a customized personal digital assistant (PDA) or a smartphone 132. (A smartphone is a mobile phone with advanced computing ability that, generally, facilitates bi-directional communication and data transfer. Smartphones include, for example, iPhones™, available from Apple Inc., Cupertino, Calif.; BlackBerries™, available from RIM, Waterloo, Ontario, Canada; or any mobile phones equipped with the Android™ platform, available from Google Inc., Mountain View, Calif.) The smartphone 132 may communicate with the system 100 using a connection already built into the phone, such as a Wi-Fi, Bluetooth, or near-field communication (NFC) connection. Alternatively, a smartphone dongle (i.e., a special hardware component, typically equipped with a microcontroller, designed to mate with a corresponding connector on the smartphone) may be used to customize the data-transfer protocol between the smartphone and the drug delivery system 100.

The functional components of drug delivery systems as described above may be packaged and configured in various ways. In some embodiments, the drug pump device may be integrated into a patch adherable to the patient's skin. Suitable adhesive patches are generally fabricated from a flexible material that conforms to the contours of the patient's body and attaches via an adhesive on the backside surface that contacts a patient's skin. The adhesive may be any material suitable and safe for application to and removal from human skin. Many versions of such adhesives are known in the art, although utilizing an adhesive with gel-like properties may afford a patient particularly advantageous comfort and flexibility. The adhesive may be covered with a removable layer to preclude premature adhesion prior to the intended application. As with commonly available bandages, the removable layer preferably does not reduce the adhesion properties of the adhesive when removed. In some embodiments, the drug pump device is of a shape and size suitable for implantation. For example, certain pump devices in accordance herewith may be used to deliver drug to a patient's eye or middle ear.

FIG. 1B shows an exemplary configuration of a drug delivery system 140 including a piston pump device 141 and an associated tethered infusion set 142, both mounted to skin-adhesive patches 143. The pump device 141 includes a cylindrical (or, more generally, tubular) vial 144 with a piston 145 movably positioned therein and an electrolysis electrode structure 146 mounted to one end. The structure 146 may be made of any suitable metal, such as, for example, platinum, titanium, gold, or copper. In another embodiment, the structure 146 may include a support made from plastic or glass containing the electrodes inside a sealed pump chamber 147. In some embodiments, the pump chamber is sealed at the back with a puncturable, self-sealing plug (e.g., made of rubber), facilitating insertion of a fill needle to inject electrolyte into the pump chamber 147, as explained further below. The piston 145 separates the interior of the vial 144 into a drug reservoir 148 and the pump chamber 147. A cannula 149 connects the drug reservoir 148 to the infusion set 142. The piston pump device 141 is enclosed in a protective housing 150, e.g., made of a hard plastic.

FIGS. 1C-1E depict another exemplary drug delivery system 160 that integrates a piston pump device 161 and lancet-insertion assembly 162 in a parallel arrangement in a single housing or device shell 163. The insertion assembly 162 includes a serter housing 164, needle carrier 165, and needle/catheter double-spring insertion mechanism 166, and is disposed above a prefilled piston pump device 167 (formed from a cartridge containing the drug reservoir and further including an electrolysis pump) and fluidically connected therewith via tubing 168. A carrier 169 provides a base for the piston pump device 167, and a connector 170 for the insertion assembly 162. The serter housing 164 holds the needle 171 and a catheter 172 (e.g., made of Teflon) that connects thereto, as well as the two springs 173 (for insertion of the needle and catheter), 174 (for subsequent retraction of the needle), and connects to the catheter hub 175. The piston pump device 167 may be contained in a pump casing 176, which, together with the insertion assembly 162, is enclosed in the outer device shell 163.

In its initial position prior to insertion, the needle 171 and catheter 172 are located above the catheter hub 175. To insert the catheter 172 into the subcutaneous tissue, a trigger button 177 is activated (e.g., manually or via an electronic signal from a system controller, such as controller 118) to release the initially compressed insertion spring 173. This moves the needle 171, needle carrier 165, and catheter 172 (hereafter the “needle carrier assembly”) downward, inserting the needle 171 with the catheter 172 through a self-sealing silicone plug 178, and into the subcutaneous tissue (FIG. 1E). The self-sealing silicone plug 178 may have two septums (top and bottom layers), providing an open area between the two layers with which the outlet of the fluid tubing 168 fluidically communicates. During insertion, the needle carrier assembly is propelled downward by the spring 173, and is stopped when the front (i.e., downward-facing in the figure) face of the needle carrier 165 encounters the rear (upward-facing) face of the catheter hub 175. The catheter hub 175 may have angled sides, which act as latches, holding the retraction spring 174 (which is still compressed) in place. The retraction spring 174 is at least as stiff as, and typically stiffer than, the insertion spring 173; thus, when released, it can compress the insertion spring 173 and drive the needle carrier assembly back into its original position. The retraction spring 173 may be released manually, e.g., when the user compresses the sides of the catheter hub 175 with thumb and forefinger. Alternatively, it may be released by an electromechnical component responsive to a release signal from the system controller. As a result of the release of the retraction spring 173, the needle 171 is extracted out of the tissue as the needle carrier assembly is driven back into the retracted position. When the needle 171 is retracted, radial and axial compression on the silicone plug 178 causes the small puncture to close immediately, providing a tight seal for the fluid path in the infusion set. Following catheter insertion, the lancet insertion assembly 162 and outer shell 163 may be removed, leaving only the pump 161 and infusion set on the skin.

FIG. 1F illustrates yet another embodiment of a drug delivery system in accordance herewith. In contrast to the wearable drug pump embodiment depicted in FIG. 1B, the system 180 of FIG. 1F is a handheld prefilled pen-injector designed for one-time use. The pen-injector 180 includes a prefilled glass vial 181 fitted with a piston 182 and electrolysis pump structure 183. Further, the pen-injector includes microelectronics 184 implementing the system controller 118 and associated circuitry, a digital dial 185 for setting a desired dosage, a digital display 186 for communicating the dosage setting to the user, and an injection button 187 for triggering release of the dosage. The various components are arranged linearly, in a “pen” configuration with the injection button 186 at the end. The pen injector 180 may be fitted with a removable needle 188, which is, prior to use during storage, protected by a suitable needle cap 189 (and, optionally, a second, outer cap 190). In certain embodiments, the needle and vial structure are disposable, whereas the back portion of the pen injector including the microelectronics 184, dial 185, display 186, and injection button 187 are re-usable. In alternative embodiments, the entire pen-injector is intended for disposal after use.

All of the drug pump device embodiments described above may utilize a pre-filled vial or cartridge fabricated from glass, polymer, or other materials that are inert with respect to the stability of the drug and, preferably, biocompatible. Glass is commonly used in commercially available and FDA-approved drug vials and containers from many different manufacturers. As a result, there are well-established and approved procedures for aseptically filling and storing drugs in glass containers, which may accelerate the approval process for drug pump devices that protect the drug in a glass container, and avoid the need to rebuild a costly aseptic filling manufacturing line. Using glass for the reservoir further allows the drug to be in contact with similar materials during shipping. Polymer vials, e.g., made of polypropylene or parylene, may be suitable for certain drugs that degrade faster when in contact with glass, such as protein drugs. Suitable glass materials for the vial may be selected based on the chemical resistance and stability as well as the shatterproof properties of the material. For example, to reduce the risk of container breakage, type-II or type-III soda-lime glasses or type-I borosilicate materials may be used. To enhance chemical resistance and maintain the stability of enclosed drug preparations, the interior surface of the vial may have a specialized coatings. Examples of such coatings include chemically bonded, invisible, ultrathin layers of silicone dioxide or medical-grade silicone emulsions. In addition to protecting the chemical integrity of the enclosed drugs, coatings such as silicone emulsions may provide for easier withdrawal of medication by lowering internal resistance and reducing the amount of pressure needed to drive the piston forward and expel the drug.

In various embodiments, the pump 104 includes a sparking mechanism for quickly recombining the electrolysis gas to relief pressure, in some cases achieving near-vacuum in the pump chamber. When the pressure in the pump chamber falls below that of the drug reservoir 102, a force is created that pushes the piston towards the back of the pump and away from the reservoir outlet. Thus, gas recombination may generate suction at the reservoir outlet and downstream thereof, which may be used during retraction of the catheter or needle following drug injection to prevent any residual drug contained in the catheter or needle from dripping into the subcutaneous tissue. This is important, for example, for intramuscular injections, where drug released into the subcutaneous tissue (rather than muscle tissue) can cause considerable harm to the patient. Suction may also be used, in some embodiments, to draw fluid (e.g., blood) from the patient into a reservoir of the pump device. For example, following injection of a volume of liquid drug into the patient, the empty reservoir (or a separate reservoir of another pump fluidically connected to the injection site) may be used to hold a blood sample for subsequent analysis.

Suction pressure can generally be achieved with any of a variety of controlled or uncontrolled gas-recombination mechanisms, including (but not limited to) spark mechanism utilizing, e.g., capacitive-discharge ignition, inductive-discharge ignition, or transistor-discharge ignition. FIG. 1G illustrates a mechanism for creating an electrical-discharge spark in the pump chamber to induce a rapid gas-recombination ignition process. Herein, a discharge arc is created simply by application of a high voltage across a gap between two wires 191 of a spark plug 192 disposed in the pump chamber 193. The spark decreases the activation energy between gas-phase hydrogen and oxygen (like a chemical catalyst) to form liquid-phase water, causing the gases to recombine virtually instantaneously. The phase change from gas-phase hydrogen and oxygen to liquid-phase water can drastically decrease the volume of the substance (e.g., by a factor of about a thousand), and this sudden volume shrinkage provides the pressure relief in the chamber 193. Recombination induced by spark ignition is very fast, usually resulting in nearly complete pressure relief (e.g., a drop down to 1% of the original pressure) within the microsecond to millisecond range. Unlike spark ignition in a combustion engine, which causes gas expansion, spark ignition to induce gas recombination causes a volume decrease; consequently, there is no risk of explosion. Further, only minimal heat is produced during the process, likewise not presenting any safety risk.

In some embodiments, the speed and/or volume reduction of gas recombination is reduced in a controlled manner to adjust the suction at the reservoir outlet. One way to accomplish controlled, reduced recombination is to shorten the ignition time of the spark, e.g., with a high-speed circuit that can quickly turn the spark on and off. By shutting down the spark, recombination can be deliberately stopped before all the hydrogen and oxygen gases have recombined. Another way to slow down spark-ignition recombination is to use a separator, such as a sold wall with a valve or a membrane, to divide the interior of the pump chamber into two compartments, one adjacent the piston and the other one containing the electrolysis electrodes as well as the spark gap. Only gas in the latter compartment will recombine and reduce the compartment pressure to zero (or nearly zero); the gas mixture in the other compartment will gradually diffuse through the compartment separation (on times scales much longer than the duration of the spark) until pressure equilibrium is reached. Via the volume ratio between the two compartments, the end pressure can be set. Of course, spark timing and compartment separation can also be used in combination in order to optimize recombination control. An alternative approach to controllable pressure relief involves releasing gas from the pump chamber through an active release valve, which may be controlled, e.g., electro mechanically or piezoelectrically by the pump's control circuitry. By closing the valve before all gas has escaped, the end pressure can be controlled. To avoid ejecting electrolyte during the pressure-relief stage, the electrolyte may be soaked into a highly absorbent material (e.g., a hydrogel). Various alternative pressure-relief mechanisms are described in more detail in U.S. patent application Ser. Nos. 13/680,828, 13/680,869, 13/680,990, and 13/681,008 (all filed on Nov. 19, 2012), which are hereby incorporated herein by reference in their entirety.

### 2. Multi-Reservoir and Multi-Pump Pump Assemblies

Various embodiments hereof incorporate multiple drug pump devices to facilitate combination drug therapies, which require the dosing of two or more (separately stored) drugs or other agents, to occur seamlessly without the need for multiple needle injections. In some embodiments, a main drug is stored in a standard reservoir, and a secondary drug is stored in a second reservoir that is part of a second pump device of equal or smaller size than the first pump device. Depending on the particular therapy, the drugs may be delivered separately to alternate between doses, or simultaneously so that they are mixed and delivered together. Combination drug therapies are widely used, for example, in cancer treatment. Further, many drugs currently under development or newly approved come in lyophilized (i.e., dry) form since they are not stable in liquid form over an ordinary shelf life. Lyophilized drugs require reconstituting the liquid drug formulation just prior to delivery. A pump with multiple reservoirs or storage chambers enables automating the process of reconstituting and mixing the drugs, eliminating the multiple steps and many pieces of equipment traditionally needed.

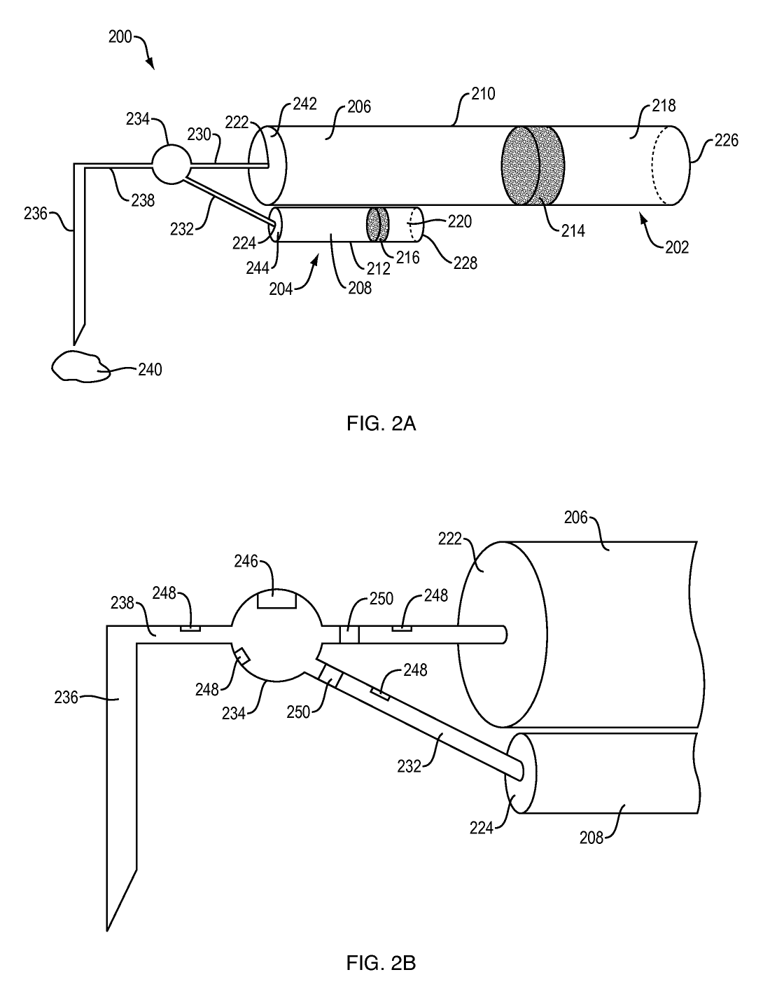

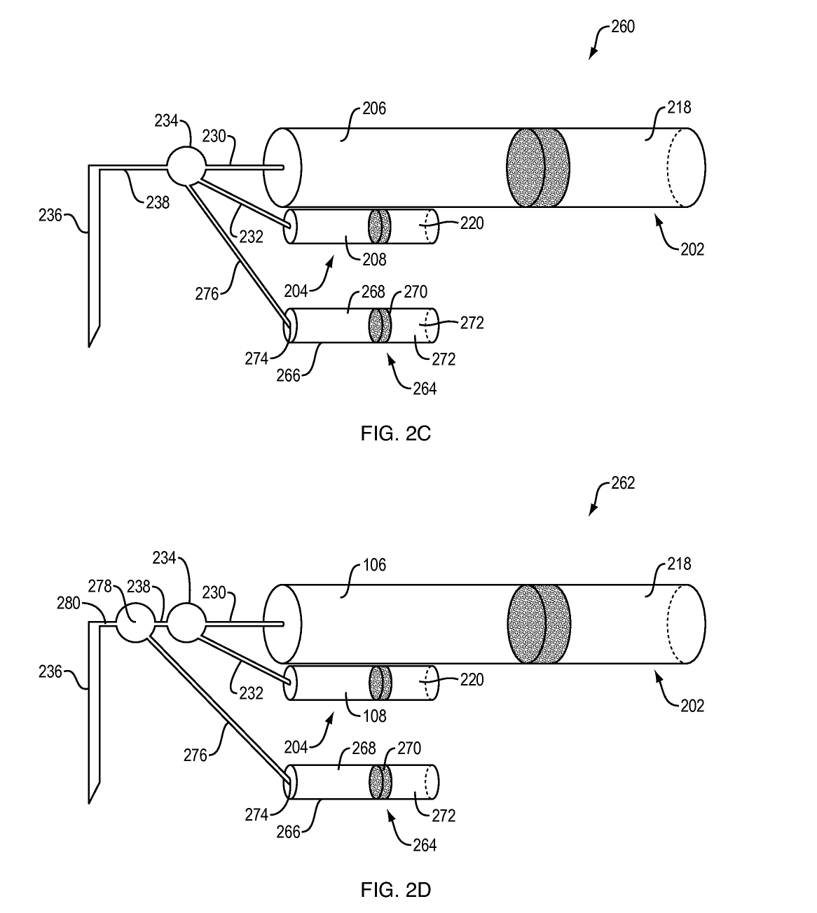

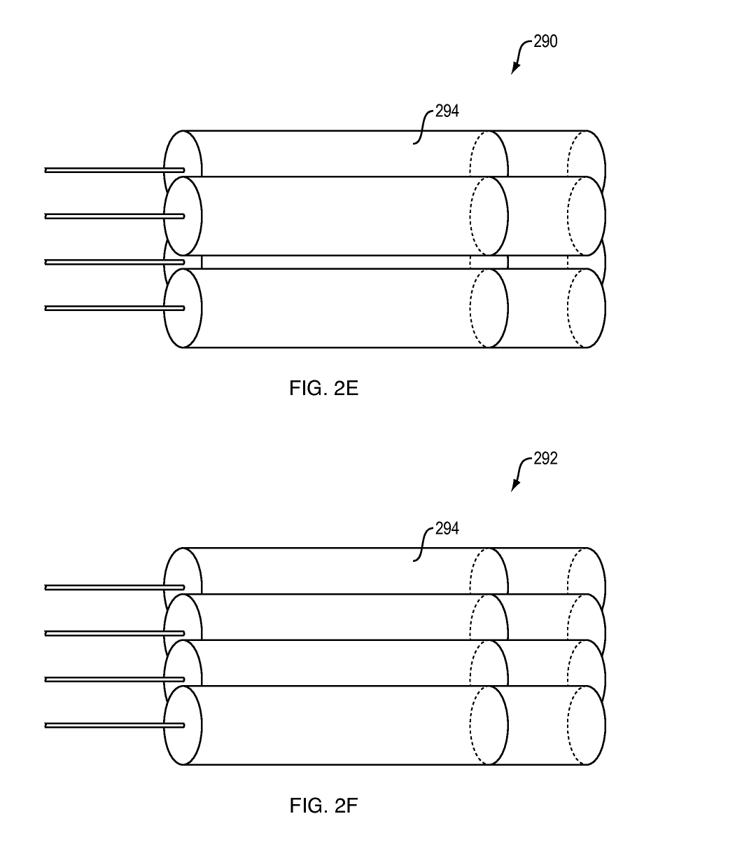

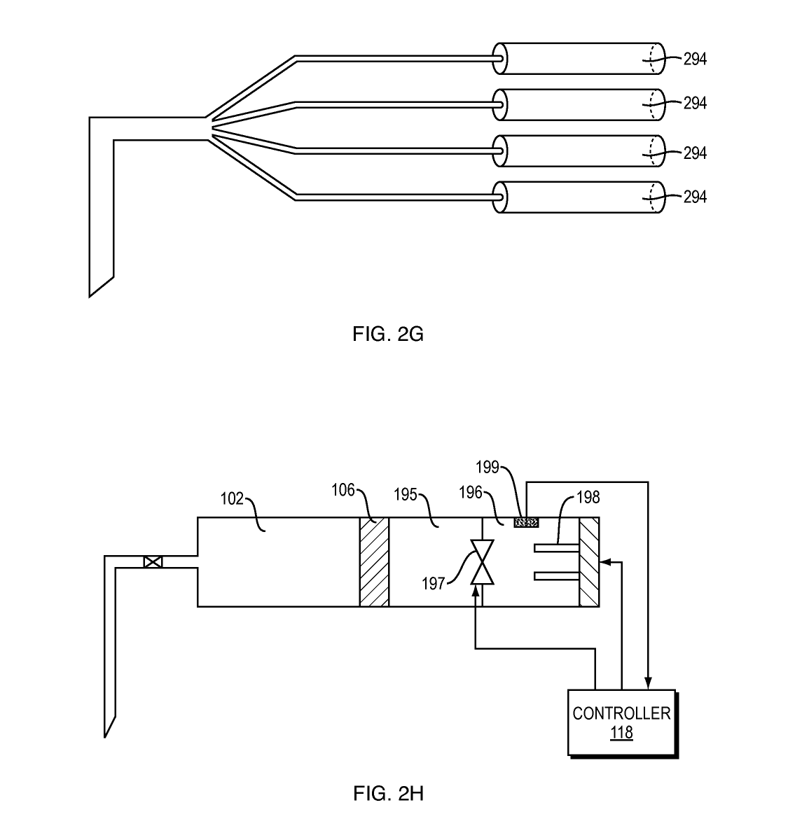

FIG. 2A depicts an exemplary drug pump assembly 200 including two drug pump devices (e.g., piston pump devices) 202, 204, which may be enclosed in a single housing or separately enclosed in two protective housings made of, e.g., a hard plastic. The pump devices 202, 204 may be configured like the devices described above with respect to FIGS. 1A-1F, i.e., each pump device 202, 204 may include a drug reservoir 206, 208 within a cylindrical (or, more generally, tubular) vial 210, 212, and a piston 214, 216 movably positioned in the respective vial 210, 212. The pistons 214, 216 separate the drug reservoirs 206, 208 from respective pump chambers 218, 220, and pressure in the pump chambers 218, 220 actuates the pistons so as to force drug stored in the reservoirs 206, 208 through respective reservoir outlets 222, 224. The pumping mechanisms may be the same or different for the two pump devices 202, 204. In some embodiments, the pump chambers 218, 220 are conventional electrolysis pump chambers filled with liquid electrolyte. As gaseous electrolysis products are generated, they push the pistons 214, 216 towards the outlet 222, 224 of the drug reservoir 206, 208, thereby expelling the drug.

The drug pump devices 202, 204 further include fluid conduits 230, 232 fluidically connecting the outlets 222, 224 of the drug reservoirs 206, 208 to a downstream mixing chamber 234, which is then connected to a drug delivery device 236 via a fluid conduit 238 that delivers drug from the mixing chamber 234 to the infusion site 240. In one implementation, the conduits 230, 232, 238 forms a “Y connector.” As used herein, the term “connecting” broadly encompasses both a direct connection between two components and an indirect connection via one or more additional, intervening components. The fluid conduits may be made of flexible tubing, bores in needles and other rigid structures, channels within a bulk structure, or, generally, any structure defining a fluidic path.

In some embodiments, each of the reservoirs 206, 208 contains a different therapeutic agent in liquid form. This allows for the separate administration of two different drugs in a staged or alternating fashion. For example, the pump chamber 220 of the first device 202 may apply a pressure to the reservoir 208 and force the first therapeutic fluid stored in that reservoir 208 out of the outlet 224 and through the tubing 232, 238 and delivery device 236 to the infusion site 240. Independently, pressure in the pump chamber 218 of the second device may force the different therapeutic fluid stored in the second reservoir 206 out of the outlet 222 and through the tubing 230, 238 and delivery device 236 to the infusion site 240. (As depicted, the fluid also traverses the mixing chamber 234. In embodiments where the two therapeutic fluids are delivered separately, this mixing chamber 234 is generally not needed. However, it may be used advantageously to mix each individual therapeutic fluid, e.g., to reverse any precipitation that may have occurred during storage.) Because each drug pump 202, 204 may be individually controlled, the therapeutic dosage of each of the different drugs may be optimally delivered to the patient based on a suitable therapeutic protocol. In some embodiments, the operation of the two pumps is controlled by a single system controller (e.g., controller 118), or by separate controllers in communication with each other and/or both receiving commands from the same higher-level controller, according to a unified delivery protocol that coordinates the dosing of the two drugs. Thus, by utilizing two drug pumps 202, 204, alternating doses of different drugs may be appropriately employed to treat different maladies.

An exemplary application context that requires alternating injections of two (or more) different drugs is chemotherapy for the treatment of brain tumors. A combination of bevacizumab (e.g., Avastin®) and CPT-II can be extremely effective in adult patients suffering from recurrent malignant glioma or in pediatric patients having high-risk malignant brain tumors. More specifically, Avastin® and CPT-II combination therapy has demonstrated rapid clinical and radiographic improvement in patients with relapsed malignant glioma. Some patients have even achieved long-term improvement. MRI scans of recurrent-glioma patients treated with Avastin® and CPT-II (as well as with carboplatin and etoposide) have shown rapid tumor shrinkage. Accordingly, in one embodiment hereof, the drug pumps 202, 204 may be employed to pulse boluses of each drug to the brain tumor at different intervals (e.g., Avastin® on odd days and CPT-II on even days). Since Avastin® and CPT-II work in different fashions (i.e., Avastin® slows down blood vessel growth by inhibiting vascular endothelial growth factor (VEGF), a protein that plays a major role in angiogenesis and in the maintenance of existing blood vessels throughout the life cycle of a tumor, while CPT-II disrupts nuclear DNA by inhibiting topoisomerase I, an enzyme that relaxes super-coiled DNA during replication and transcription), pulsing boluses of each drug at different intervals allows the drugs to work without interfering with each other. In addition, steroids may be pulsed intermittently with the Avastin® or CPT-II to aid the surrounding brain edema during tumor treatment.

In some embodiments, the drug pumps 202, 204 are employed to deliver a combination of two drugs simultaneously. For example, two different isoforms of an anti-vascular endothelial growth factor (anti-VEGF) may be employed to treat age-related macular degeneration. To ensure that the two drugs are well-mixed prior to injection, the drug pump system 200 may include the mixing chamber 234 to temporarily store and/or mix the two streams of the first and second therapeutic fluids. The mixing chamber 234 may be a passive chamber that allows the two drugs to mix via diffusion. Alternatively, referring to FIG. 2B, the mixing chamber 234 may include a stirring means 246 (e.g., a pump, fan, turbine, or magnets) for actively stirring and mixing the fluid contained therein. In one embodiment, the stirring means 246 is controlled by the system controller 118 based on readings from one or more sensors 248 located in the mixing chamber 234 and/or fluid conduits 230, 232, 238. For example, when the sensor(s) 248 detects that the second therapeutic fluid is flowing into the mixing chamber 234, the system controller 118 may send a command to the stirring means 246 to mix the fluids. Once the combined fluids are well-mixed in the chamber 234, they may be delivered to the target site 240 through the delivery device 236. The combined fluid may be delivered from the mixing chamber 234 to the infusion site 240 while both the first and second therapeutic fluids are being released from the reservoirs 106, 108 to the mixing chamber 234. In this case, the stirring means 246 may be deactivated when the delivery of the combined fluid is completed and/or there is no fluid flow in the conduit 238. Alternatively, the first and second therapeutic fluids may be expelled to the chamber 234 during a first time interval, and mixed in the mixing chamber 234 during a second time interval. After the mixing is complete, the combined fluid is delivered during a third time interval. In this case, the stirring means 246 may be deactivated upon detecting that the combined fluid has begun to flow out of the mixing chamber 234 (e.g., beginning of the third time interval). In addition, the stirring time (i.e., length of the second time interval) may depend on the properties (e.g., viscosity or mixability) of the therapeutic fluids. In general, the sensor(s) 248 may be flow, thermal, time of flight, pressure, or other sensors, as are well-known in the art.

In some embodiments, one or more flow-regulator structures (e.g., valves) 250 are deployed in the conduits 230, 232 that fluidically connect the outlets 222, 224 of the reservoirs 206, 208, respectively, to the mixing chamber 234, for the purpose of controlling the delivery of the fluid(s) and/or preventing backflow. The flow-regulator structure(s) 250 may be positioned at or near the distal ends (i.e., proximal the mixing chamber 234) of the conduits 230, 232. Alternatively, the flow-regulator structure(s) 250 may be positioned elsewhere along the length of the conduits 230, 232, such that the ends are proximal to the reservoirs 206, 208, respectively. In various embodiments, the reservoirs 206, 208 and/or the conduit 238 may include one or more such flow-regulator structures instead of, or in addition to, the flow-regulator structure(s) of the conduits 230, 232.

In one embodiment, the flow-regulator structures 250 include one or more check valves that are normally closed such that fluid cannot pass through; this prevents forward flow of the drug until sufficient pressure is generated by the drug pumps 202, 204. When fluid pressure in the conduits 230, 232 exceeds a predetermined threshold value (i.e., a cracking pressure), the check valves open and allow fluid to flow from the reservoirs 206, 208 to the mixing chamber 234. Accordingly, when a check valve in one of the fluid paths is closed, a high flow pressure may build up in this path before the fluid is released; this may be particularly useful when, for example, a high delivery flow rate of released fluid using the built-up pressure is desired. Additionally, because the check valves remains closed when the fluid pressure inside the conduits 230, 232 is equal to or less than the fluid pressure in the more distal conduit 238, the fluid is prevented from flowing backwards into the drug reservoirs 206, 208.

In another embodiment, the flow-regulator structures 250 include one or more active valves to actively regulate the fluid flow, for example, to maintain a constant flow rate. In this way, the administered dosage of the drug depends on the duration that fluid containing the drug flows through the conduits 230, 232, rather than on the magnitude of an applied pressure that drives fluid flow through the conduits 230, 232. Additionally, the active valve(s) may operate in conjunction with the system controller 118 to perform closed-loop flow control. More accurate control of the administered dosage may thereby be obtained, and the dosage remains independent of external mechanical influences (e.g., a force applied by the patient). The active valves may be manufactured and operated using any conventionally available approaches; see, e.g., U.S. Pat. Nos. 8,246,569 and 7,090,471, the entire disclosures of which are hereby incorporated by reference.

Valves may also be used to control mixing of multiple therapeutic fluids in the mixing chamber. For example, valves in the fluid conduits 230, 232 from the drug reservoirs 206, 208 may be open until the mixing chamber 234 reaches a certain fill level (which may, e.g., be measured directly, or inferred from a measured flow rate through the valves 230, 232 as integrated over time), at which point the valves 230, 232 close. A valve 250 between the mixing chamber 234 and the delivery device 236 remains closed for a specified period of time and/or until a desired degree of uniformity has been achieved in the mixture. Then, the valve 250 opens, allowing the mixed fluids to be injected into the patient. In fact, using valves 230, 232, 250 in this manner, passive mixing can, in principal, be achieved in a section of the fluid conduit therebetween, without the need for a separate chamber. The chamber 234, however, facilitates storage and mixing of larger fluid volumes, and may further include active mixing means, as described above.

In various embodiments, one of the therapeutic fluids includes a secondary agent affecting the pharmacokinetics at the injection site; this agent may be applied before, during, or after delivery of the primary drug. For example, injecting epinephrine into the local area of the infusion site 240 (e.g., subcutaneous is tissue or intramuscular tissue) may affect the local blood vessels (e.g., causing a constriction), thereby slowing down the dispersion of the primary drug injected at a later time. Because reducing the diffusion or absorption of the primary drug may prevent the drug from being processed by the body, using the agent is particularly useful in oil-based intramuscular drugs, which are generally taken up rapidly by the body. Similarly, in some embodiments, one of the therapeutic fluids may include an activating agent that activates the primary therapeutic fluid. The activating agent and primary drug may be mixed in the mixing chamber 234 prior to injection into the patient. Further, a dual-pump system in accordance herewith may also find application in clinical trials, where one of the fluids may include the therapeutic agent while the other fluid may serve as a control. Typically, the control fluid is physiologic saline. The saline solution may be given prior to injecting the test therapeutic fluid; a response to the physiologic saline may then serve as a controlled response for analyzing the effects of the test therapy. Accordingly, utilization of two drug pumps 202, 204 in unison leads to various medical advantages, including, for example, the ability to affect the local infusion environment pharmacokinetically, activate a primary drug, and/or provide a controlled response for clinical trials.

While the drug pump system 200 illustrated in FIG. 2A has only two pumps 202, 204, multi-pump devices that combine three or more pump devices may also be manufactured for use in certain applications. For example, referring to FIGS. 2C and 2D, pump systems 260, 262 including three pumps 202, 204, 264 may be employed to deliver appropriate amounts of one, two, or three types of drugs. In some embodiments, the third drug pump 264, like the other two pumps, includes a cylindrical vial 266 having a drug reservoir 268 therein, and a piston 270 movably positioned in the vial 266, separating the drug reservoir 268 from a pump chamber 272. Again, the pump chamber 272 may utilize any pumping mechanism (e.g., the same mechanism as employed by either one or both of the other pumps 202, 204, or a different mechanism) to actuate the piston 270, thereby forcing the drug stored in the reservoir 268 through an outlet 274 thereof. Further, the third drug pump 264 includes a fluid conduit 276 to expel the drug from the reservoir 268. Referring to FIG. 2C, the fluid conduit 276 may connect the outlet 274 to the downstream mixing chamber 234, which then releases the drug to a target site 240 via the drug delivery device 236. Again, the mixing chamber 234 allows the fluids from the reservoirs 206, 208, 268 to be well-mixed before being injected into the patient. Alternatively, referring to FIG. 2D, the fluid conduit 276 may connect the outlet 274 of the reservoir 268 to a second mixing chamber 278 downstream of the mixing chamber 234 and upstream of the drug delivery device 236. As a result, fluids leaving the first mixing chamber 234 and fluids from the third drug pump 264 both flow to the second mixing chamber 278. A fluid conduit 280 then connects the second mixing chamber 278 to the drug delivery vehicle 236 for releasing the drugs. The third pump 268 may be utilized to inject an additional drug, a pharmacokinetic agent, a drug-activating agent, or a control fluid for clinical trials.

Referring to FIGS. 2E and 2F, the drug pump devices of multi-pump systems may be arranged and configured in different ways. In various embodiments, each individual drug pump device 294 is manufactured using its own vial, which forms a drug reservoir therein, contains the piston, and is fitted with a pump for actuating the piston. The vials (and, consequently, drug pumps) may be all of the same size or vary in size (particularly length). Regardless, different vials may store the same or different types and/or volumes of drug. As shown in FIG. 2E, the pumps 294 may be stacked to form multiple layers, e.g., in a dense-packing configuration in which each pump in a higher layer is placed above the “valley” created between two pumps in the lower layer; this configuration minimizes the overall cross-section of the system and may be used, e.g., in multi-pump pen injectors. Alternatively, as shown in FIG. 2F, the pumps 294 may be arranged next to each other in a single layer to achieve a low device profile. Other configurations and arrangements may be suitable for different systems and applications. The reservoir outlets of the pump reservoirs may be connected via fluid conduits to one or more mixing chambers, which are in turn connected to a drug delivery device, as indicated in FIGS. 2A-2D. Each pump 294 may optionally include one or more sensors and/or one or more flow-regulator structures as describe above. Operations of the multiple pumps 294 may be controlled by one or more controller 118. In some embodiments, all of the pumps 294 and, optionally, the associated tubing, delivery device, and/or control circuitry are enclosed within a single housing and/or integrated into a single skin patch.

As described above, drug delivery systems with multiple drug pumps may be used to administer different therapeutic fluids in a seamless manner. The multi-pump systems do, however, also find applications for the delivery of a single therapeutic fluid. For instance, referring to FIG. 2G, multiple drug pumps 294 arranged in parallel may be operated simultaneously or sequentially to increase the total flow rate of drug or the total volume of drug delivered to the target site while utilizing fixed-size components, such as standard-size vials. For example, certain standard vials store up to 3 ml of drug. For therapies that require drug volumes in excess of that amount, multiple such vials may be combined; to achieve a total volume of 12 ml, for example, four pumps may be activated.

Further, in drug therapies that involve multiple bolus injections of the same drug, drug pumps may be sized to store each specified bolus amount in a separate vial. The vials may be integrated into one system and may all be connected to the same delivery needle or catheter, facilitating multiple injections with one needle insertion. Each bolus delivery involves simply activating one of the pumps until the reservoir contained therein is empty. In other words, the system controller 118 may individually operate the drug pumps 294 to sequentially release the entire specified dosage of the fluid drug stored in the reservoir of each pump 294, e.g., based on a programmed delivery protocol. Advantageously, because the bolus amounts are separated in different reservoirs, this method completely avoids overdosing any individual injection (provided that the individual pump does not store an excessive amount of drug), in contrast to a larger pump device that stores a drug volume sufficient for many injections in a single reservoir, where delayed pump shut-off or residual pump pressure can easily result in an injection exceeding the desired amount. Using multiple drug pumps therefore offers the advantage of multi-dosing and safe injections without a risk of overdose.

In certain embodiments, drug pump devices are provided with multiple pump chambers in a serial arrangement to achieve higher pressures and, thus, faster drug delivery. FIG. 2G. shows, for example, an electrolytically driven syringe pump device with a single reservoir 102 and two adjacent pump chambers 195, 196, which are separated, e.g., by a wall including an active valve 197. The electrolysis electrodes 198 are contained within the chamber 196 at the back of the device (away from the piston), whose volume is fixed. During pump operation, the valve 197 is initially kept closed, confining the generated electrolysis gas to the fixed-size pump chamber 196 to build up pressure therein. Once the pressure in the chamber 196—as measured, e.g., with a pressure sensor 199 disposed in the chamber 196—reaches a certain threshold value, the valve 197 is opened such that the electrolysis gas can flow into the pump chamber 195 adjacent the piston and move the piston forward. The initial pressure applied to the piston following opening of the valve 197 depends on the volume ratio between the two chambers 195, 196, and on the pressure built up in the back chamber 196, which, in turn, is a function of the electrical current supplied to the electrodes 198 and the duration of electrolytic gas generation. Electrolysis and valve operation may be controlled by the system controller 118 in response to a pressure signal received from the sensor 199. While the pressure in the electrolysis chamber 196 quickly drops once the valve 197 is opened (e.g., to half of its original value if the chamber 195, 196 are of equal size), the built-up pressure can, in some embodiments, vastly exceed the pressures achievable in a pump device having only a single pump chamber that is contact with a movable piston and, thus, expands as gas is generated. Accordingly, multi-pump-chamber devices are useful for fast injections of drug boluses. With devices configured for multiple bolus injections, the time period between successive boluses is available for building up pressure in the electrolysis chamber 196. With devices intended for one-time use, such as a pen injector, the pump may be turned on to build up pump pressure before the needle is inserted into the patient so as to minimize the time that the needle remains inserted. Of course, a multi-chamber pump device may also be used for continuous drug delivery by simply leaving the valve 197 open.

### 3. Drug Pump Device Manufacture, Filling, and Preparation for Use

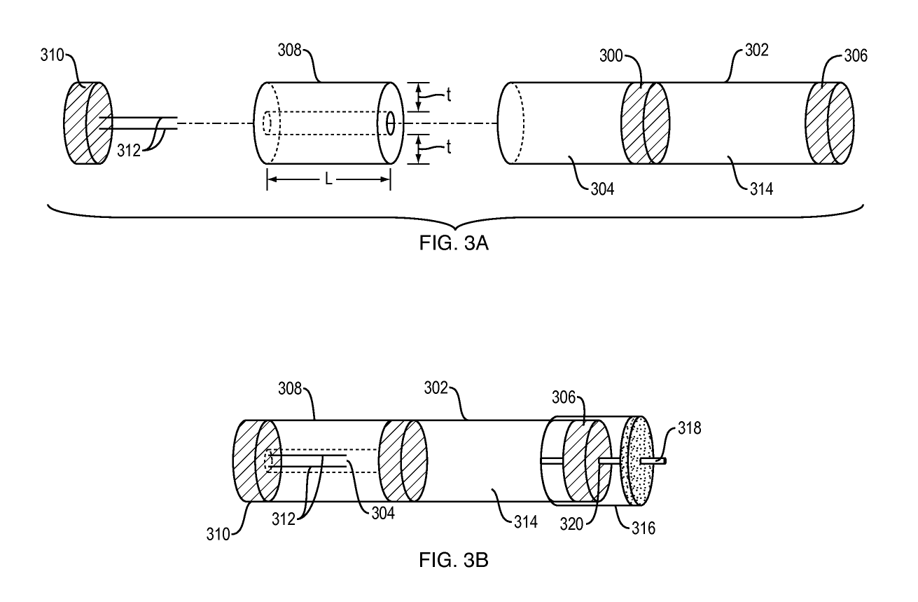

In certain embodiments, electrolytic piston pump devices are manufactured by fitting a conventional, commercially available glass or polymer drug vial, which may already be validated for aseptic filling, with the piston and electrolysis pump components. (Alternatively, to accommodate the pump chamber, the vial may be longer than typical commercially available vials, but maintain all other properties such that validated filling methods and the parameters of existing aseptic filling lines need not be changed.) Referring to FIGS. 3A and 3B, the piston 300 may be disposed inside the vial 302 near one end, leaving room for the pump chamber 304, and a septum 306 may be disposed at the other end to seal the vial 302. Both the piston 300 and the septum 306 may be made of an elastomeric polymer material, such as a synthetic or natural rubber; in some embodiments, silicone rubber is used. A spacer 308 may be inserted into the vial 302 at the pump end to prevent the piston 300 from sliding too far back during assembly. The spacer 308 may, e.g., take the form of a cylindrical ring wall whose outer diameter is fitted to the interior of the vial 302; the piston 300 may be positioned against the rim of that ring wall. The pump assembly 310, in turn, may rest against the other end of the spacer 308. The pump assembly 310 generally forms the back wall of the vial 302 and carries the electrolysis electrodes 312 (which may extend into the pump chamber 304 defined between the piston 300, spacer 308, and pump assembly 310); in various embodiments, it also includes the pump controller, battery, and associated circuitry, e.g., housed within, integrated with, or attached to the back wall.

The length l of the cylindrical-wall spacer 308 may be chosen, depending on the length of the vial 302, to achieve a desired size of the drug reservoir 314 formed between the piston 300 and septum 306: the longer the spacer 308, the shorter is the reservoir 314. This way, a drug vial of standard length can easily be adjusted to accommodate different volumes of liquid drug. The wall thickness t of the cylindrical-wall spacer 308 may be selected based on its length l to achieve a desired initial pump chamber volume and, thereby, affect the pump pressure resulting from a fixed amount of electrolysis gas. To achieve similar pump characteristics for pump devices with reservoirs of different size (within same-size vials), the spacer wall t is generally the thicker, the longer it is. Of course, the spacer 308 need not be shaped like a cylinder wall. In some embodiments, it is simply a solid cylinder inserted between the piston 300 and pump chamber 304, acting, in effect, like an extension of the piston 300. In these embodiments, pump pressure is exerted onto the spacer 308, which transfers it to the piston 300; spacer 308 and piston 300 then move together to expel drug from the reservoir 314. In other embodiments, the spacer 308 includes openings (such as the central bore in the cylindrical-ring-wall configuration, or multiple bores through an otherwise solid cylinder) that provide a fluidic path between the electrolysis electrodes 312 and the piston 300, allowing the electrolysis gas to push the piston 300 directly. The spacer 308 may, in this case, be fixedly positioned relative to the vial 302, allowing electrolysis gas to fill the space between the piston 300 and the spacer 308 as the piston 300 moves forward. The spacer may be made of hard plastic, glass, metal, or some other solid material, and generally serves simply to reduce the volume between piston 300 and pump assembly 310 that is available to the gas, thereby increasing the pump pressure generated per unit of electrolysis gas produced. In some embodiments, the pump assembly 310 and spacer 308 are integrated into a single structure that can be snapped in place inside the vial 302.

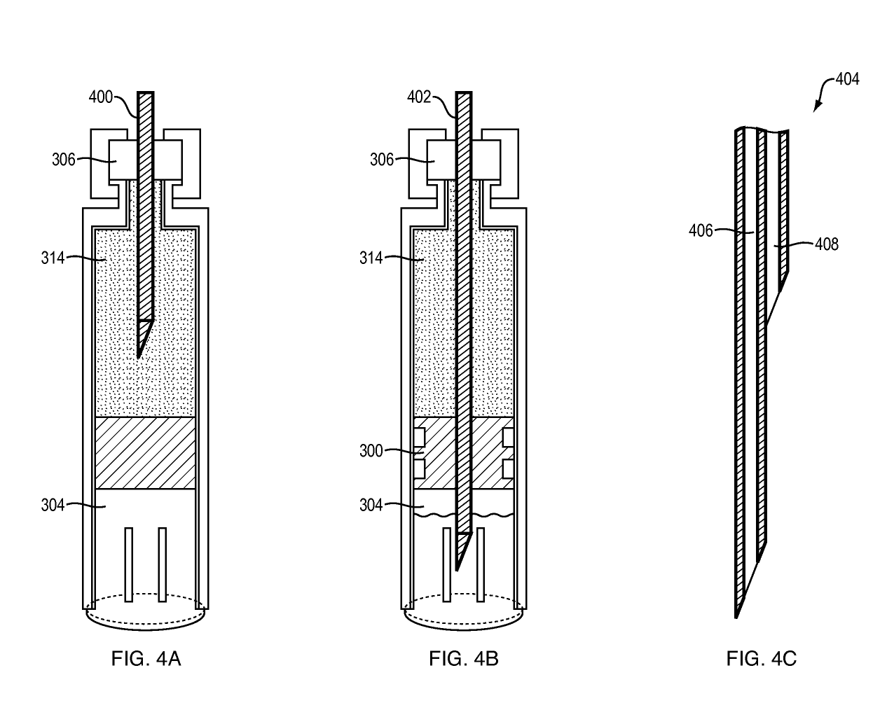

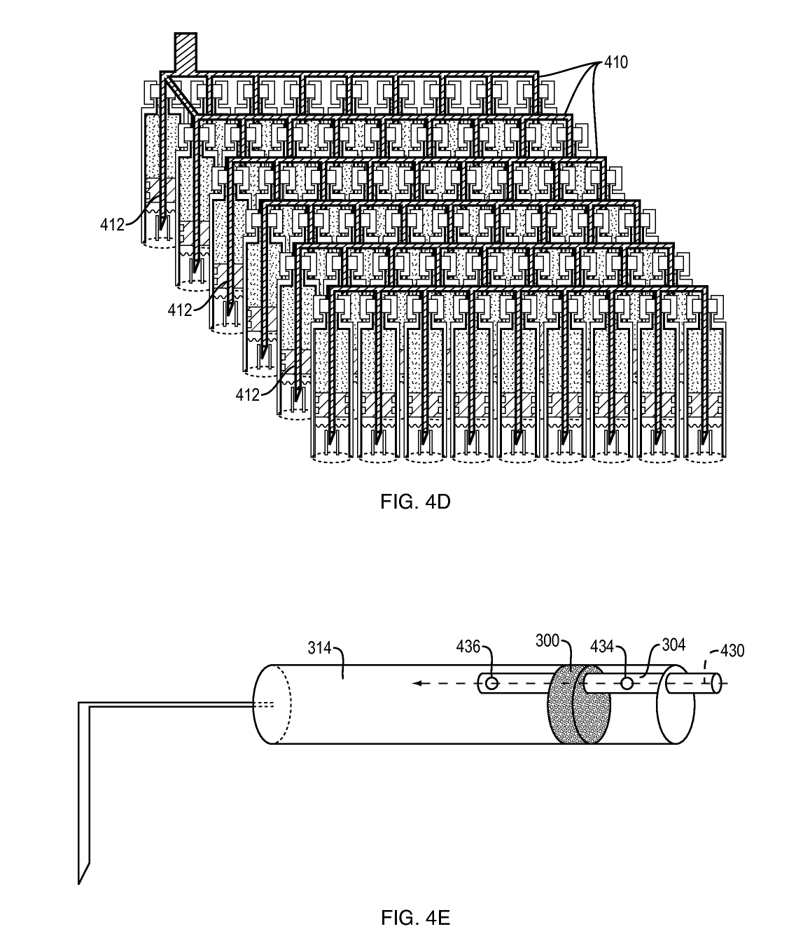

Following assembly of the piston pump device and sealing of both ends (via the septum 306 and pump assembly 310, respectively), the device may be filled, i.e., liquid drug may be injected into the reservoir 314 and electrolyte into the pump chamber 304. As shown in FIGS. 4A-4C, this may be accomplished with a needle 400 that pierces the septum 306 to deliver the drug to the reservoir 314, and the same or another needle 402 that pierces both the septum 306 and the piston 300 to reach the pump chamber 304 in order to fill it with electrolyte 404. (If the same needle is used, it may first be inserted into the drug reservoir to inject liquid drug, and thereafter be further advanced to puncture the piston 300 and inject electrolyte into the pump chamber 304.) In some embodiments, two needles 400, 402 are advanced into the reservoir 314 and pump chamber 304, respectively, at the same time to allow the reservoir 314 and pump chamber 304 to be filled simultaneously, saving time. In place of two independently movable needles, a single needle structure 404 with two co-axial bores 406, 408 of different lengths may be used for this purpose. After filling of the drug pump device, the needle (or needles) may be pulled out, allowing the septum 302 to self-seal and, thus, once again, close the device at the front end.

FIG. 4D illustrates a filling system for a multi-chamber drug delivery device (as described above, e.g., with respect to FIGS. 2C-2F). The system includes a fluidic manifold 410, comprising interconnected tubing, that routes fluid from a common fill reservoir (e.g., of the liquid drug or the electrolyte) into the different devices. The branches of the fluidic manifold end, for each device, in a needle structure 412 of sufficient length and sharpness to pierce through multiple septa to gain access, e.g., to the pump chambers.

In some embodiments, the piston pump device is filled from the back rather than through the septum 306 at its front end. For example, the back wall, or a portion thereof, may be made of the same or a similar material as the septum 306 and/or piston 300, or some other self-sealing pierceable material, allowing the pump chamber 304 to be filled via a needle inserted through the back wall and allowing the drug reservoir 314 to be filled via a needle penetrating both the back wall and the piston 300. As with needles 400, 402 inserted through the septum 306, two needles may be integrated into a single structure that facilitates simultaneous drug and electrolyte injections. Alternatively, as shown in FIG. 4D, the drug pump device may include a permanent fill port 430 penetrating both the back wall 432 and the piston 300. The fill port 430 may have two bores with respective exit orifices 434, 436 placed in the pump chamber 304 and reservoir 314. The fill port 430 may be affixed to the vial 302 and pump assembly 310 and allow the piston 300 to slide along the port 430. In alternative embodiments, the fill port is affixed to and/or integrated with the piston 300, which does not only ensure that the orifice 436 remains located in the drug reservoir 314 as the piston 300 moves, but also provides a means for moving the piston 300 forward manually, by pushing the portion of the fill port 430 that extends beyond the back end of the device, in case normal pump operation fails. Entering the drug reservoir 314, for filling purposes, via the pump chamber 304 may be advantageous in that it keeps the septum 306, through which the drug will ultimately be expelled, intact during the manufacturing process. During later drug delivery, a screw-in needle cassette 316 placed over the septum 306 may be screwed (e.g., using a mechanical actuation mechanism) into the vials 302 such that the cassette needle 318 punctures the septum 306, creating a reservoir outlet 320 and establishing a fluid connection with the cannula.

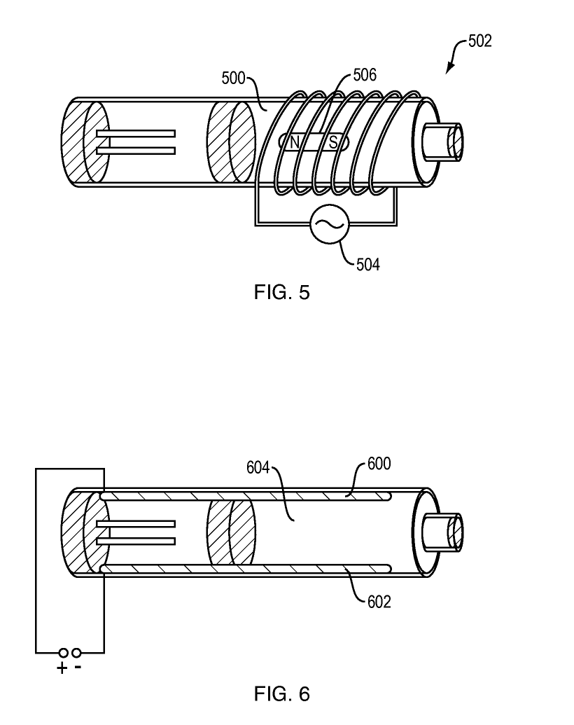

Prefilled drug pump devices in accordance with various embodiments are intended for long-term storage, targeting shelf lives of up to two or three years. Even if the individual chemical constituents are stable, drug formulations may suffer, over such long time periods, from precipitation and/or segregation of their different components (such as multiple therapeutic agents). Accordingly, they may require mixing prior to injection. To obviate the need for the patient (or health care provider) to shake the device and/or to achieve better, more uniform distribution of all components of the mixture, various drug pump devices utilize an electrically powered magnetic stirring mechanism. For example, as shown in FIG. 5, the drug reservoir 500 may be surrounded by an electromagnetic sleeve or coil 502 connected to an alternating current (AC) source 504, which creates, upon activation, a varying magnetic field within the reservoir 500. The reservoir may contain a magnetic stirrer 506, e.g., a rod-shaped permanent magnet, that rotates within this field, thereby mixing the contents of the reservoir. Certain drug formulations include nano-magnetic particles that create fluid currents in response to electromagnetic fields; these and other ferric suspensions do not, or not necessarily, need a stirrer 506 to get properly mixed. Other magnetic or non-magnetic stirring mechanisms may also be used. For instance, a motorized rotating stirring rod, fan, or similar structure may be mounted inside the reservoir. Magnetic stirring mechanisms are, however, often preferred due to the mechanical isolation they allow between the stirrer and the walls of the reservoir, which preserves the integrity of the reservoir.

Some drugs require a certain pH range (e.g., low pH, high pH, or a pH of about 7) for storage. However, the pH of a therapeutic solution may also affect its rate of absorption, and the optimal pH for storage is not always also optimal for delivery. Various drug pump embodiments in accordance herewith address this discrepancy by changing the pH within the reservoir prior to delivery, e.g., using, as shown in FIG. 6, a pair of electrodes 600, 602 disposed in the drug reservoir 600 (e.g., threaded in the interior wall of the vial). A voltage applied between the electrodes 600, 602 can cause an electrochemical reaction that alters the pH. For example, H⁺ may be generated at the anode 600 to lower the pH, and OH⁻ may be produced at the cathode 602, raising the pH. In addition to the shelf life and absorption characteristics of a drug, its ability to be delivered using certain routes of administration may depend on the pH. Drugs to be taken orally, for example, are preferably acidic so as to match the environment of the stomach, whereas drugs formulated for subcutaneous or intramuscular injection should have a relatively neutral pH (e.g., in the range from 6 to 8). The ability to change the pH of the therapeutic solution before delivery may, therefore, also facilitate formulating drugs for alternate routes of administration, potentially resulting in better therapies. Further, a change in pH may be used, in some embodiments, to activate a drug, i.e., to change its chemistry for treatment efficacy. In general, electricity applied in the drug reservoir may serve to alter the chemical or physical environment in various ways to improve storage and delivery conditions. For example, the cathode and anode may catalyze a reaction to activate the drug, or cause gas development that effectively changes the drug concentration in the liquid.

### 4. Lancet Insertion and Pain Reduction

Many drug pump embodiments described herein are designed to enable patients to self-administer the drug. In this context, it is particularly important to provide drug delivery devices that are safe and easy to use, as well as to minimize any discomfort or pain associated with the drug injections. Therefore, various embodiments hereof are directed to improved lancet insertion devices and mechanisms.

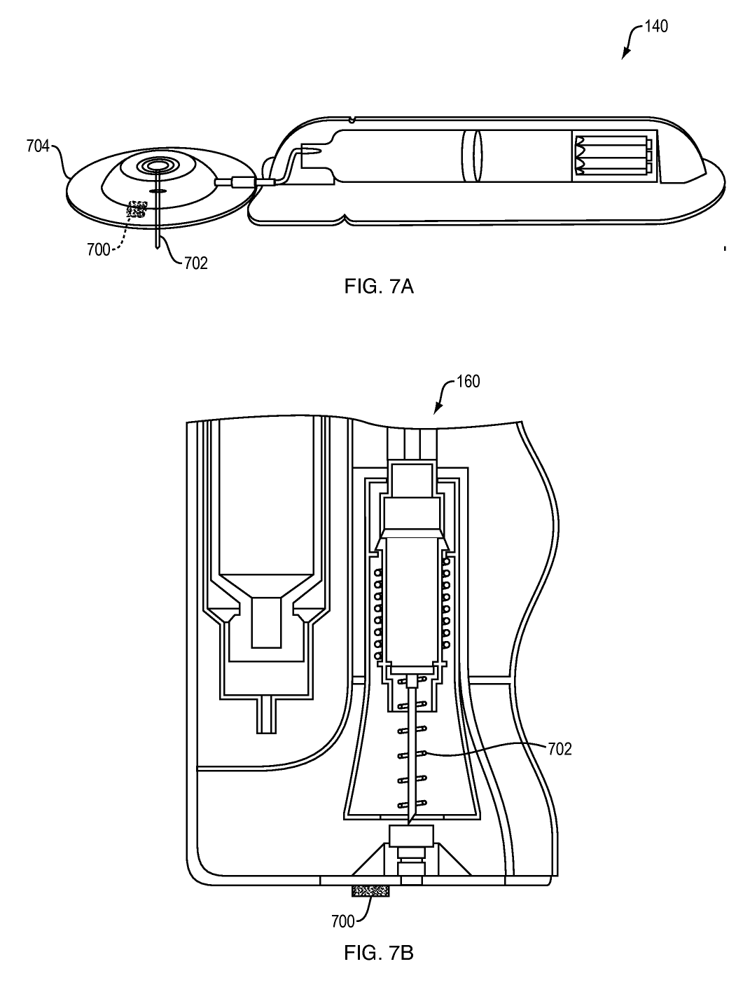

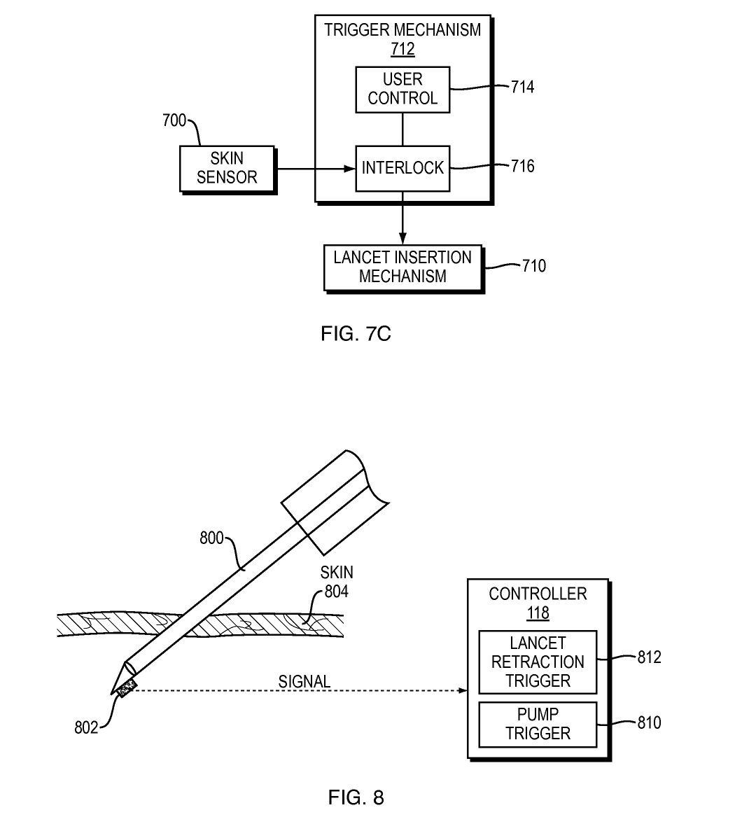

In some embodiments, a skin sensor is used to check whether the drug delivery device (and, in particular, the component responsible for lancet insertion) is properly placed in contact with the skin and ready for injection. As show in FIGS. 7A and 7B, the skin sensor 700 may be placed on the bottom of the device, in close proximity to the exit port through which the lancet or needle 702 is expelled. In a patch pump device, the sensor 700 may be integrated with or placed on the adhesive patch 704, as shown in FIG. 7A. For a handheld or other device with a hard shell or housing, the sensor may be integrated into or attached to the housing, as shown in FIG. 7B. The sensor may measure impedance or temperature to detect the presence of skin, exploiting the large difference of these parameters between air and skin. For instance, the average temperature of the skin surface is about 34° C., whereas standard room temperature is 25° C. The resistivity of dry skin is around 10²-10⁴ Ohm-m, whereas it is on the order of 10¹⁶ Ohm-m for air. Once the impedance sensor measures a resistance within the average range of skin resistance, or the temperature sensor measures skin temperature, it can enable the user to trigger the lancet, either by directly enabling the lancet insertion mechanism (e.g., a mechanism as described above with respect to FIG. 1E, or any other suitable mechanism) and/or by enabling a trigger mechanism including a user-operable control for allowing the user to trigger lancet insertion. In other words, the sensor may act as a safety interlock, disabling the lancet insertion mechanism until the device has skin contact. Thus, inadvertent triggering of the lancet by the user is largely prevented, which reduces the risk of injuries during handling of the device.

FIG. 7C illustrates the interrelation between the various functional components of an exemplary system for triggering lancet insertion only when skin contact is detected. The system includes a lancet insertion mechanism 710 and a trigger mechanism 712, responsive to the skin sensor 700, for activating the lancet insertion mechanism 710. The trigger mechanism 712 includes a user-operable control 714, such as a button to be pushed by the user (e.g., button 177) to inject the lancet, as well as a safety interlock 716 that receives input from the skin sensor 700. The safety interlock 716 may be implemented electronically (in hardware and/or software), e.g., via a module of the system controller 118, and may electronically trigger the lancet insertion mechanism 710 if, and only if, the sensor 700 measures skin contact and the user actuates the control 712. Alternatively, the user control 714 may be configured to mechanically trigger the lancet insertion mechanism, and the safety interlock 716 may include a mechanical component (e.g., a piezoelectric coupler) that mechanically decouples the user control 714 from the lancet insertion mechanism 712, or otherwise effectively deactivates the trigger mechanism 712, unless it receives a signal indicative of skin contact from the sensor 700.

In some embodiments, illustrated in FIG. 8, the lancet or needle 800 is equipped, at or near its tip, with a sensor 802 that detects when the skin 804 has been pierced. This determination may be based on a measured pH, temperature, conductivity (e.g., to detect contact with sweat), or some other parameter indicative of the current sensor location in or relative to the tissue. The sensor 802 may be or include a capacity sensor, magnetic sensor, electrical sensor, pH sensor, or microphone (which also detects vibrational forces), or any other suitable sensor type, and may have the ability to discriminate between different types of tissue so as to ascertain, for example, whether the lancet tip is inside the subcutaneous tissue or a muscular layer as desired, or whether it has hit, e.g., a nerve or vein. The sensor signal may be sent to the system controller 118 or electronic circuitry 124, e.g., via a dedicated wire or, in some embodiments, via the needle (which is typically made of metal and, thus, electrically conductive) itself. The sensor feedback may then be used as input for controlling the drug delivery device. For example, in some embodiments, the pump is automatically activated (e.g., by turning on the drive current) when the skin has been pierced. As another example, upon detection that the needle has hit a vein or nerve, pump operation may be stopped and a needle retraction mechanism may be triggered automatically and immediately. Trigger mechanisms 810, 812 responsive to the sensor 802 for initiating pump and/or terminating pump operation and for causing needle retraction (e.g., using the retraction mechanism described above with respect to FIG. 1E), respectively, may be implemented electronically (in hardware and/or software), e.g., as modules of the system controller 118 and/or software modules stored within the system memory 126).

Automating needle insertion (and, if necessary, retraction) may help minimize the mechanical components that the patient needs to operate, which does not only render the device easier to use, but may also facilitate (e.g., as a result of the increased ease of use) proper needle insertion due to a better grasp of the device. Inserting the needle properly may, in turn, limit the discomfort and/or pain felt by the patient during insertion. For instance, if the pump is automatically started once the needle is in the skin, the patient need not depress an additional button to initiate the drug injection. This may free his second hand, which the patient can now use, e.g., to pinch the skin or align and stabilize the needle for steady insertion, both of which reduce pain and discomfort. Similarly, in pen injectors designed to be held and operated with one hand, elimination of a trigger button for activating the pump may allow the patient to change his grip on the pen injector, increasing his dexterity and, consequently, ability to hold the needle at approximately the optimal insertion angle and/or advancing it at approximately the optimal speed. For example, for needles with beveled tips, pain is generally minimized by a quick stab at an approximately 45° angle with the bevel tip facing down (i.e., toward the skin in the direction of insertion). A quick, confident stab can be difficult to achieve if the patient needs to use his index finger or thumb to manually cause drug injection by pressing a button at the end of the pen injector, but can be accomplished more easily if automatic pump operation allows the patient to hold the pen injector securely between his thumb, index finger, and middle finger. A proper grip of the handheld injector also serves to reduce any motion of the needle tip while inserted in the subcutaneous tissue (or muscle), which likewise contributes to increased comfort.

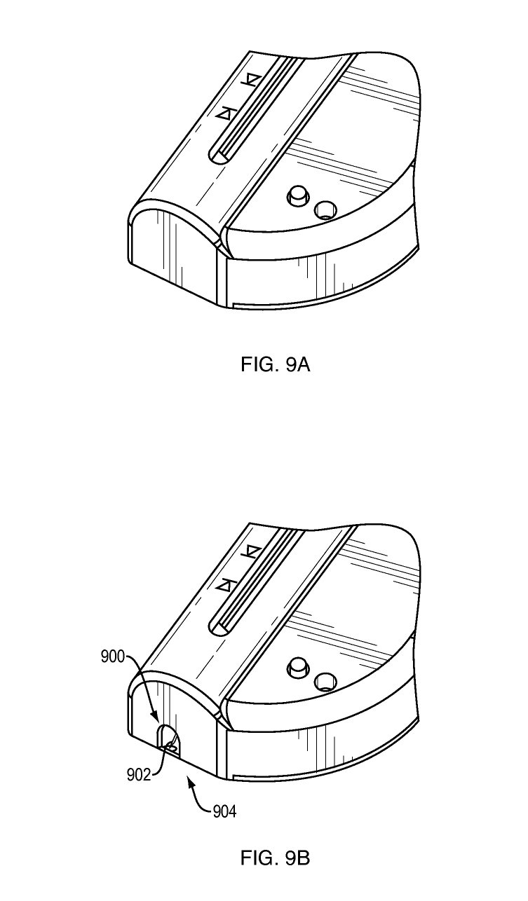

In certain drug delivery device embodiments, such as the integrated delivery system 160 depicted in FIG. 1C, view of the injection site is ordinarily obstructed by the device housing (or related components), as illustrated in FIG. 9A. To remedy this problem, the housing may feature a window 900 (shown in FIG. 9B), formed either by a transparent material or simply the absence of any material in a certain region, that allows observations of the injection site. In the device of FIG. 9B, the needle 902, whose tip can be seen through the window 900, is oriented at about 45° and exits the device through an opening in the bottom 904 of the case. This allows the patient or healthcare provider to notice, for example, if blood accumulates inside or around the cannula (e.g., due to piercing of a blood vessel), and reposition the device, or throw it away and use a new device, as necessary.

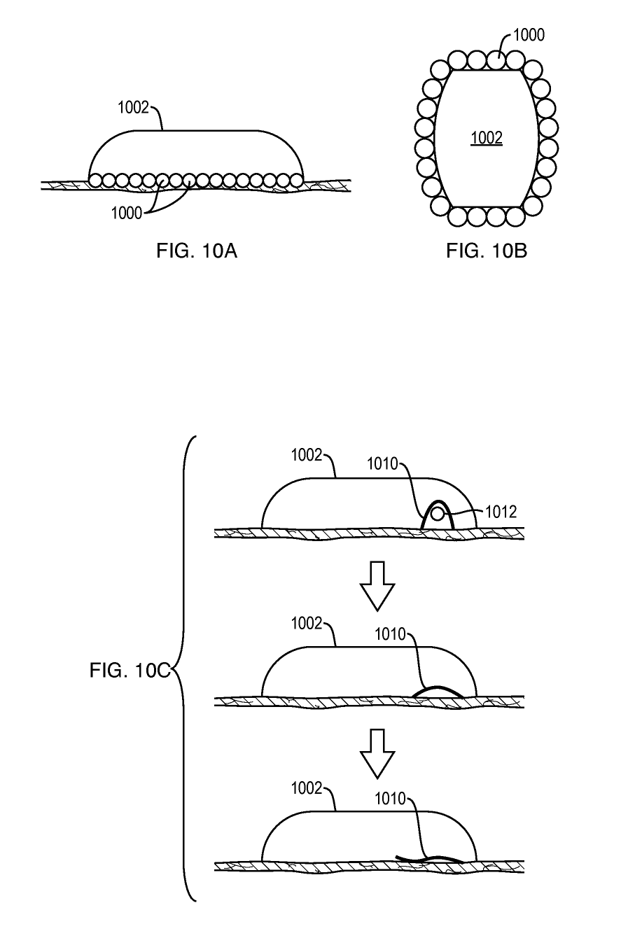

In some embodiments, drug delivery devices in accordance herewith incorporate a mechanism for distracting the patient immediately before needle injection in order to reduce the perceived pain associated therewith. In one embodiment, illustrated in FIGS. 10A and 10B, a vibrator structure (including one or more vibrating rings 1000 or motors) is placed on or integrated with the housing 1002, e.g., at a location that is, in use, in contact with the skin. (This embodiment is relevant, in particular, to patient-worn patch pump devices.) Alternatively, a vibrational chip may be used to vibrate the entire device, vibrations of the housing portions in contact with the skin being felt by the patient. The vibrational chip may, generally, be positioned anywhere inside the device; in some embodiments, it is integrated with the circuit board implementing the system controller and/or related circuitry. An alternative distraction mechanism, shown in FIG. 10C, utilizes one or more rubber bands 1010 (or other type of strands) that are initially under tensile stress and, upon release, snap against the patient's skin to cause a minor sensation of pain that distracts from the subsequent needle injection. Various suitable stress and release mechanisms are well-known to those of skill in the art and can be selected and implemented without undue experimentation; examples include a lever or latch 1012 that releases the band 1010. As will be readily appreciated by persons of skill in the art, the patient may be distracted by many alternative mechanisms, including, e.g., a pinch, a dull prick, or the sudden release of cold water (e.g., from a small, cooled water reservoir contained in the pump).

Whatever the distraction mechanism, its timing is, in accordance herewith, coordinated with the timing of the piercing of the skin by the needle. To effectively distract the patient from the needle insertion, the distraction mechanism preferably operates one second or less before the needle is inserted. This relative timing may be achieved by suitable electronics that provides signals to trigger first the distraction mechanism and, shortly thereafter, the needle insertion mechanism (which may, e.g., include the release of an insertion spring as described above with respect to FIG. 1E). Alternatively, the distraction mechanism and needle insertion mechanism may be mechanically coupled. The distraction mechanism may, for example, trigger the needle insertion mechanism via a series of latches.

The terms and expressions employed herein are used as terms and expressions of description and not of limitation, and there is no intention, in the use of such terms and expressions, of excluding any equivalents of the features shown and described or portions thereof. Rather, having described certain embodiments of the invention, it will be apparent to those of ordinary skill in the art that other embodiments incorporating the concepts disclosed herein may be used without departing from the spirit and scope of the invention. For example, while this disclosure describes piston pump devices and, in particular, electrolytically driven piston pumps, many aspects described herein may also be used with other pump configurations (including diaphragm pumps as described, e.g., in U.S. patent application Ser. No. 12/875,266, filed on Sep. 3, 2010.) and other types of pumps (e.g., electrochemical, osmotic, piezoelectric, pneumatic, or motor-driven pumps). In fact, many aspects and features described herein, particularly those relating to needle insertion and pain reduction as well as to mixing the drug and/or changing its chemical environment in the reservoir, are largely agnostic to the particular pump configuration and pump mechanism employed. Further, while certain features have been described herein with respect to either only portable drug pump devices or only pen injectors, and while these features may be particularly advantageous in the embodiments for which they have been described, this is not to be understood as limiting the applicability of these features to only those embodiments. Rather, embodiments of the invention may possess any or all of the features and advantages described herein, in any suitable combination, even if such combinations were not made express herein. Accordingly, the described embodiments are to be considered in all respects as only illustrative and not restrictive.

What is claimed is:

1. A drug pump assembly comprising:
two piston pump devices, each comprising (i) a vial having a drug reservoir therein, (ii) a piston movable within the vial for forcing drug out of an outlet of the reservoir, and (iii) a pump for actuating the piston;
a first mixing chamber downstream of the reservoirs; and
first fluid conduits connecting the outlets of the reservoirs with the first mixing chamber and a second fluid conduit connecting the first mixing chamber with a drug delivery vehicle downstream thereof.

2. The assembly of claim 1, further comprising a third piston pump device comprising (i) a vial having a drug reservoir therein, (ii) a piston movable within the vial for forcing drug out of an outlet of the reservoir, and a (iii) pump mechanism for actuating the piston.

3. The assembly of claim 2, further comprising a third fluid conduit connecting the outlet of the third reservoir of the third piston pump device with the first mixing chamber.

4. The assembly of claim 2, further comprising a second mixing chamber downstream of the first mixing chamber and upstream of the drug delivery vehicle and a third fluid conduit connecting the outlet of the third piston pump to the second mixing chamber, the second fluid conduit connecting an outlet of the first mixing chamber to the second mixing chamber and connecting the second mixing chamber to the drug delivery vehicle.

5. The assembly of claim 4, wherein at least one of the first mixing chamber or the second mixing chamber comprises a stirring mechanism.

6. The assembly of claim 5, wherein the stirring mechanism comprises at least one of a pump, a fan, a turbine, or magnets.

7. The assembly of claim 1, further comprising at least one valve between at least one of the reservoir outlets and the first mixing chamber.

8. The assembly of claim 7, wherein the at least one valve comprises a check valve preventing backflow.

9. The assembly of claim 7, wherein the at least one valve comprises an active valve regulating fluid flow.

10. The assembly of claim 9, further comprising:
at least one sensor disposed within at least one of the drug reservoirs or fluid conduits for monitoring at least one parameter therein; and
a controller for controlling the at least one valve based on the monitored parameters.

11. The assembly of claim 1, wherein the pump comprises at least one of an electrochemical pump, an osmotic pump, an electro-osmotic pump, a piezoelectric pump, a thermo-pneumatic pump, an electrostatic pump, a pneumatic pump, an electro-hydrodynamic pump, a magneto-hydrodynamic pump, an acoustic-streaming pump, an ultrasonic pump, or an electrically driven mechanical pump.

12. A method for treating a target using an assembly comprising two piston pump devices, each comprising (i) a vial having a drug reservoir therein, (ii) a piston movable within the vial for forcing drug out of an outlet of the reservoir, and (iii) a pump for actuating the piston, the method comprising:
actively mixing liquids released from the drug reservoirs of the two piston pump devices in a mixing chamber; and
delivering the mixed liquid to the target via fluid conduits.

13. The method of claim 12, further comprising monitoring at least one parameter of the liquids in the piston pump devices.

14. The method of claim 13, further comprising regulating flows of the liquids based on the monitored parameter.

15. The method of claim 12, further comprising creating a negative pressure in at least one of the piston pump devices so as to prevent the mixed liquid from infiltrating the target or induce the mixed liquid to flow in a direction from the target site to the piston pump devices.

16. The method of claim 15, wherein the pump is an electrolysis pump generating electrolysis gas within a pump chamber in mechanical contact with the piston, and wherein negative pressure is created in the pump chamber using a mechanism for recombining the electrolysis gas.

17. A method for treating a target using a drug pump assembly comprising two drug reservoirs fluidically connectable to an injection site in the target, the method comprising:
providing a first therapeutic fluid from a first one of the reservoirs to the target; and
subsequently providing a second therapeutic fluid, different from the first therapeutic fluid, from a second one of the reservoirs to the target,
wherein the first therapeutic fluid pharmacokinetically affects a local environment of the target and the second therapeutic fluid comprises an active ingredient for treating the target.

18. A method for treating a target using a drug pump assembly comprising two drug reservoirs and a mixing chamber, the method comprising:
delivering a first therapeutic fluid from a first one of the reservoirs to the mixing chamber;
providing a second therapeutic fluid, different from the first therapeutic fluid, from a second one of the reservoirs to the mixing chamber;
mixing the first and second therapeutic fluids in the mixing chamber; and
delivering the mixed first and second therapeutic fluids to the target,
wherein the first therapeutic fluid comprises an active ingredient for treating the target and the second therapeutic fluid activates the active ingredient of the first fluid.

19. A clinical trial method using a drug pump assembly comprising two drug reservoirs, the method comprising:
delivering a therapeutic fluid from a first one of the reservoirs to a target within a patient and measuring a response of the target thereto;
delivering a physiological saline solution from a second one of the reservoirs to the target and measuring a response of the target thereto; and
comparing the responses of the target to the therapeutic fluid and the physiological saline solution, respectively, to thereby determine an effect of the therapeutic fluid.

20-60. (canceled)
